(19) United States
(12) Patent Application Publication
(10) Pub. No.: US 2004/0230257 A1
(43) Pub. Date: Nov. 18, 2004

(54) Enhanced bioavailability of nutrients, pharmaceutical agents, and other bioactive substances through laser resonant homogenization or modification of molecular shape or crystalline form

(76) Inventor: Todd F. Ovokaitys, Carlsbad, CA (US)

Correspondence Address: RANDALL B. BATEMAN, BATEMAN IP LAW GROUP, 4 TRIAD CENTER, SUITE 825, PO BOX 1319, SALT LAKE CITY, UT, 84110, US

(21) Appl. No.: 10/774,746

(22) Filed: Feb. 9, 2004

Related U.S. Application Data
(60) Provisional application No. 60/446,146, filed on Feb. 10, 2003.
(60) Provisional application No. 60/505,910, filed on Sep. 25, 2003.

(51) Int. Cl. A61N 1/00

(52) U.S. Cl. 607/88

(57) ABSTRACT

A method for improving the bioavailability of a bioactive substance includes subjecting the bioactive substance to laser radiation. The laser radiation modifies the bioactive substance to thereby modify reactions relating thereto in the body. The method enables reductions in inflammation associated with autoimmune diseases, modification of reaction by-products in the body, increased homogenization and flattening of molecular shape and improved methylation. The improved methylation can be utilized to reduce homocysteine blood levels, and to reduce anxiety, depression, paranoia, hostility, somatization (perception of bodily distress) and obsessive-compulsive symptoms. Enhanced nitric oxide generation from modified L-arginine can be used to reduce systolic and diastolic blood pressure, lower total and LDL cholesterol levels, and improve the ratio of total to HDL cholesterol. Increased depth of penetration of sparse constructive nodes of laser radiation may increase the range of photodynamic therapy applications and a wide range of in vitro and in vivo modifications of molecular shape and activity. Laser acoustic resonance can be utilized to increase the homogeneity of crystals, or favor the generation of novel or preferred crystalline forms.

### RELATED APPLICATIONS

The present application claims the benefit of U.S. Provisional Application No. 60/446,146, filed on Feb. 10, 2003, and U.S. Provisional Application No. 60/505,910, filed on Sep. 25, 2003.

### BACKGROUND

1. Field of the Invention

The present invention relates to novel dietary amino acid and nutrient products, as well as enhanced pharmaceutical products, and method for producing the same. More particularly, the present invention relates to such products and a method wherein the products have advantageously modified bioactivity reaction profiles and method for producing said products by means of exposure to specific amplitude modulated and structured laser light processes. These processes alter the bond structure and shape of molecules in the compound and thus alter the reaction characteristics such that certain preferred biological reactions can be enhanced and in other cases less preferred reactions can be suppressed at least over an initial period after ingestion or administration so the product can be more accurately tailored to deliver a desired therapeutic or nutrient effect.

2. State of the Art

The body or specific organs in the body make use of nutrients in a variety of complex ways. These biological processes often occur in reactions that are moderated by enzymes. The efficiency of a given nutrient or compound depends on the relative ease with which it can be incorporated by the body in the desired form. This ease of incorporation is termed “bioavailability” for the purposes of the present disclosure. Thus, it will be understood that references to increased bioavailability may relate to the amount of a compound that is utilized by the body, or the speed or efficiency with which the compound is utilized depending on the context discussed.

Furthermore, improved bioavailability can also refer to improvements in the manner in which a compound is absorbed. In other words, absorption of a nutrient or pharmacologic agent with decreased irritation or reduced adverse effects is improved bioavailability, even though the actual amount of the compound absorbed may not increase.

It is known from Strachan PCT/GB00/03280 that sparse nodes of constructive interference of electromagnetic (EM) waves generated as rapidly as sub-femtosecond duration can be configured to overcome much of the limitations of scatter pathways through organic and other molecular media to selectively stimulate specific molecular resonances far more efficiently than ordinary laser EM stimulation.

Ordinarily, laser EM stimulation tends to rapidly degenerate to nonspecific thermal effects through the scattering medium. In contrast, the polarization and field structure of the EM radiation in a sparse constructive node beam can be maintained sufficiently stable such that the polar and hydrophobic regions of a molecule differentially absorb laser energy from these sparse constructive nodes to influence structural bond energy due to primarily acoustic resonance of the molecule altering molecular shape, and consequent chemical reactivity.

Sparse constructive nodes are generated and modulated through an optical device as described in Strachan EP865618A1. Specifically, a laser beam is passed through a first diffraction grating, a refractive element, and a second diffraction grating such that the beam is substantially canceled.

A refractive element allows the cancellation to occur over a small percentage of the wavelength variance of the laser source rather than at a single critical wavelength. This means that a complex Fresnel/Fraunhoffer zone will be generated, defined by the beat frequency of the high and low frequencies as a function of the aperture.

Thus, relatively sparse zones of constructive interference will occur between the high and low frequency passes of the cancellation element in selected directions from the aperture. Fractional changes in wavelength of the laser or relative amplitudes of wavelengths in the laser cause rapid translation in the location of these nodes. In effect the continuous beam is transformed into a string of extremely short duration pulses, typically of sub-femtosecond duration by the simple means of relatively small low frequency amplitude modulation.

Strachan (PCT/GB00/03280) also describes the use of an array of constructive node beams to sequence and promote the folding steps of a protein much as a chaperonin-like effect. In addition, amino acid structures that may have heterogeneous forms in the dry state may be homogenized into a more self-consistent form to selectively alter the biological reactivity of the structure.

In particular, the homogenization can make metabolic utilization significantly more efficient, due to the consequently simpler enzyme moderated reactions resulting from the reduced range of crystalline forms. This is especially so when the resulting forms are generally more polar, increasing the production of desired products relatively more rapidly than usual, and thereby reducing nonspecific degradation of the substrate.

Strachan further describes the ability to favor the production of structures of desired “handedness” in chiral compounds (as well as epimers through logical extension) through modulating the beam at the resonance of the structure, either to enhance the production of the desired rotation, or reduce the production of the less desired rotation. This last effect can be further promoted by the application of a rotational component to the polarization state of the beam.

### SUMMARY OF INVENTION

While Strachan teaches that it is possible to modify the structure of various compositions, it focuses principally on cell adhesion, integrins and apoptosis. The present invention, however, has found that the method discussed in Strachan is highly advantageous for the modification of amino acids, phytonutrients, nutrient and food substances, pharmacologic agents, and other bioactive substances regardless of the method of administration, to modify the bioavailability of the substance and/or to modify the manner in which the body reacts to that substance.

On a fundamental level, the present invention involves a method utilizing the technology taught in Strachan regarding molecular stimulation. In particular, a beam of light is passed through a bioactive substance in such a manner that resonance causes the modifications in the molecular structure of the molecule. This may be the folding of the molecule, the promotion or inhibition of a certain “handed-ness” of a stereoisomeric molecule, or simply a modification in the molecular dimensions of the molecule. By selectively controlling the molecules, however, significant changes can be made in bioavailability, and/or physiologic reaction to the molecule.

The intrinsic line variation of the gas laser in the absence of an etalon or other line narrowing apparatus is adequate to provide the fractional frequency shift needed to traverse the sparse constructive nodes through the mixture. The polarization plane of the laser will define the primary axis of crystalline formation or distortion in the case of a dry state application. Note that a circularly polarized laser will tend to favor the crystallization of one stereoisomer over the other. Further control of the final molecular form can be provided by modulating the laser amplitude at a frequency resonant with a given bond or bond group in the molecule or crystal.

In the absence of a specific amplitude modulation, the modulation caused by the constructive node traverse resulting from laser line instability will tend to thermally energize and spread the carbon-hydrogen and carbon-oxygen and hydrogen-oxygen bonds while leaving planar and cyclic carbon bonds at a low energy. This will have a tendency to “dry” the crystalline form in that it will tend to reduce the water content in the molecule.

There will be a tendency for stray hydrogen bonds to proliferate in the molecule at least temporarily, largely as a result of this effect. The dry state of the molecule (even in solution) will, of course, result in the crystal form being less constrained by the hydrophobic nodes and in the absence of this force the laser stimulation and thermal vibration of the outer bonds will tend to favor a flatter form of the molecule. Thus, whereas amino acid compounds and isomers typically tend to crystallize more or less randomly from various seed crystals in a solution depending on the bonded water distribution, under laser stimulation, the vast majority of the crystal formation will be of the flattest form the molecule will allow. Thus the entire “mixture” of random molecular configurations will tend to become highly homogeneous.

While it would seem that this process might best be applied during initial crystallization, in practice, this is not necessary as the laser stimulates the water bonds directly and thus can effectively “evaporate” the bonded water in a the dry state as well as alter the bond formation in a solution form. In addition to the water bond effect the asymmetric heating of the molecule combined with the field forces from the EM wave itself will induce the flatter state in any case where the modulation frequency is below 3 MHz and above 100 KHz.

Specific molecular forms can be induced by the use of specific modulation frequencies and node traverse speeds; however, this patent deals primarily with the bulk effects of laser acoustic resonance and its application to the manipulation of the physiological effects of nutritional supplements and pharmacologic agents.

In this respect one skilled in the art of physiological and pharmacological effects desired may consider which of a group of amino acids or bioactive substances he wishes to be metabolized preferentially or temporally in advance of other components of a mixture and adjust the metabolic absorption of the compound accordingly by means of the disclosed method.

The present invention relates to specific compounds that are treated according to the method disclosed with the simple intent of increasing or modifying the metabolization rate of the entire mixture. This produces the measured crystallographic effects described. The resulting physiological effect may be inferred from the bioassay results and from the clinical trials below.

Efficiency of the laser stimulation is improved if the compound is maintained at a neutral pH and is exposed at a background temperature of 25-35 degrees C. It is critically important that the laser stimulation average power is extremely low, less than would raise the bulk substrate temperature by more than a degree per mole per second, as otherwise purely random thermal effects will dominate the resonance and field effects of the laser.

The sparse constructive nodes in the optical device taught by Strachan occur as rapidly translating islands of constructive interference in a background of photons that are highly self-cancelled through destructive interference of photons in the center frequencies of the laser wavelength band. The beat frequency generated as the difference between the highest and lowest frequencies in the laser wavelength band produces constructive nodes that are very precisely placed in space. The next photon packet arrives at the same space in resonance with the traverse of the preceding photon packet. The resulting train of resonant constructive nodes behaves as though it is a series of ultrashort pulses separated spatially and temporally by an intervening medium of highly self-interfered waves. The duration of these effective pulse nodes can be as brief as subfemtosecond in their translation across molecular structure.

The impulse or “bang” on each molecule from the sparse constructive node is defined by photon absorption from the node or retransmission from the molecule that has been stimulated to ring due to acoustic resonance. Photon absorption or retransmission is described in mathematical terms as a Dirac, an impulse that functions as a spike essentially of infinite height and infinite narrowness. In order to make any structure ring at a resonant frequency, it must be stimulated with a frequency equal to or higher than its natural frequency, which condition is thus satisfied by any photon absorption or emission.

The passage of the molecule from the constructive node with a high probability of photon absorption into the much larger intervening space of the destructive nodes with a very low probability of photon absorption means that the molecule will have a high probability of releasing the photon, i.e., dropping the electron orbit of one or more of its atoms in the intervening time. Since the molecule will react to both the absorption and release of photons with an acoustic vibration on the backbone of the molecule, ideally the constructive nodes would be provided exactly at this frequency.

However, while that is the ideal, any presentation of constructive nodes substantially lower in frequency than the primary backbone resonance, but still faster than the damping time of the molecular resonance will be preferable to continuous wave laser stimulation in terms of delivering a flattening or stretching effect on the molecule. Delivering the ideal frequency is best, but just above this frequency there is likely to be interference between the constructive nodes and substrate coupled acoustic energy that would reduce the stimulation to the continuous wave laser effect, or basic thermal heating.

As an alternative to delivering the exact resonant frequency of the backbone of a given molecule, in many cases it may be better to deliver the pulse train “bang and ring” effect of a low modulation frequency sufficient to cause a rapid node translation and avoid the potential frequency overshoot of attempting to deliver a perfectly tuned wave that may degrade to a purely random thermal effect.

In the case of a general homogenization process it is possible that the inhomogeneity of the reagent would be sufficient to make a pure wave high Q delivery counterproductive, so that delivery of a pulse train frequency below the resonant frequency of the molecular backbone, yet faster than the damping time of the molecule, may in many cases give a preferable result than attempting to match the resonant frequency of the molecular backbone.

In selected cases in which more specific molecular effects are desired, the first step may be a general molecular homogenization followed by tuning the constructive node frequency to that of the backbone resonance or other specific intramolecular resonances.

The sparse constructive node frequencies may be tuned by using a wider or narrower band Strachan optical interference plate, using a primary laser with a wider or narrower emission line, adjusting the aperture or angle of the interference plate, using a higher or lower frequency laser, or modulating the primary laser beam by electronic amplitude modulation or passing the beam through an acoustical-optical crystal modulation system.

Higher frequency modulation can be achieved through a wider band interference plate, use of a laser with a wider wavelength emission line, use of a higher frequency laser, or a higher frequency of primary beam modulation before it traverses the Strachan optical interference device.

The transition of a sparse constructive node past a given point is defined by the complex interaction of the various phase additions of the beat frequency and the modulations of the beat frequency by changes in both the center frequency of the laser and the relative amplitudes and positions of the upper and lower limits of the laser emission line which straddle the interference band of the Strachan optical device.

The modulation of the laser can be quite slow even when the constructive interference nodes are translating past a fixed point in space at very high speed. Even if there were no modulation of the laser, the beat frequency of the upper and lower limits can still cause a moving rather than a standing constructive wave pattern. If the constructive node transition frequency can be anything other than zero when the modulation frequency is zero, it follows that in general the node transition frequency will be higher than the modulation frequency.

The are always several frequencies involved: the laser frequency that is uncancelled above the interference canceling frequency and the frequency that is below the cancelled frequency, the beat frequency of those as the sum and as the difference, and the spatial separation of the constructive nodes versus the speed of the phase traverse, the last depending on the aperture and the frequencies involved.

Also there is the absorption “pulse” of the transition of the electron shell as an atom absorbs or releases a photon, which can be considered infinite relative to the other frequencies.

The sparse constructive node beam differs significantly from conventional continuous wave lasers in its interactions with molecular structure. When a molecule absorbs a photon from a conventional continuous wave laser, the stimulated atom tends to remain excited with the electrons in the excited shell, because in the absence of the destructive nodes the atom is always bombarded by photons. The atom in an excited state becomes reflective of further photons. Since the atom cannot absorb more photons from a continuous beam, it is neither excited more, nor is it capable of effectively emitting photons because as soon as the electron shell tries to drop to a lower energy, another photon impinges from the beam so that the molecular structure is not excited acoustically.

In the case of a sparse constructive node beam, however, the atom absorbs a photon and the molecule rings a little as it redistributes the kinetic energy of the absorption. Photon absorption sends a pulse wave to the other end of the molecule along the backbone, that in turn reflects to the origin, so the process of absorption, travel down the backbone to the opposite end, and reflection to the origin intrinsically tends to occur at the natural frequency of the backbone, determined by the shape, size, and composition of the molecule. If the subsequent destructive node lasts long enough, then as the kinetic energy acoustic signal reflects down the backbone, it will release the photon. Once again the kinetic energy of the release will distribute along the backbone.

In the case of an ideally tuned sparse constructive node beam, as the kinetic acoustic wave hits the end of the molecule and reflects to the origin, a new constructive node will arrive at the molecule and once again excite ground state atoms to higher shells. In this resonant case with the described tuned sparse constructive node, the “shock” of the arriving photon is in phase with the ringing of the shock of the previous photon absorption and release. Hence the increase in overall kinetic energy of the molecule is now twice what it would be if stimulated by an ordinary continuous wave laser.

The process repeats as above and depending on the damping loss of the molecule, which depends on the bond structure, the kinetic energy will rise from this factor of two to a factor of many thousands. The kinetic energy or temperature of the molecule is thus raised substantially with respect to its local environment.

If the constructive nodes are too close, then the resonant buildup as described will be inhibited through lack of sufficient relaxation time for photon re-emission. Likewise the coupling of acoustic energy molecule to molecule through the medium in which the molecule exists (water if in solution and the solid if the molecule is in powder form) will tend to interfere with the above described pure resonance. Too much or too close acoustic coupling tends to have the same effect as the sparse nodes being too close and the result can be the inability of the molecule to absorb and release photons at the ideal resonant frequency reducing the ability to amplify the net kinetic energy of the molecule.

There are critical differences between the sparse constructive node effect and conventional continuous wave lasers in terms of energy transmitted to the molecule being stimulated. If the molecule is bathed in continuous wave light of the given wavelength, all of the atoms that can, will absorb photons and will have excited electrons. The hydrogen bonds will be destroyed temporarily; once that happens, the thermal effects will cause molecules in bulk to oscillate at greater amplitude but there will only be random forces on individual molecules.

Conversely in the case of sparse constructive node irradiation, the individual molecules rarely saturate with absorption of all possible photons, but rather will have time to absorb and release photons and will tend to do so at the resonant frequency of the backbone. Thus the energy exchanged with the backbone is higher in the sparse constructive node mode than in continuous wave laser mode and in addition the molecule is excited in a high polarization EM field state impossible in a highly scattered continuous wave mode.

Excitation of the molecule by sparse constructive node laser stimulation is caused by the absorption and release of photons, which absorptions and releases can be considered as infinite frequency impulses, much as the impact of a clapper hitting a bell is infinite with respect to the frequency of the bell's ring. At the same time the molecule is excited by these absorptions and releases, it is also under stress from the electric and magnetic field of the electromagnetic wave that is generally very large with respect to the stimulated molecule.

For example, L-arginine and betaine molecules are only a few nanometers long, while the laser wavelength in stimulation experiments has been 670 nanometers long. The effect can be considered similar to tapping a sheet of iron filings on top of a magnet. If you don't tap the sheet the iron filings stick to the sheet. As the sheet is tapped the filings are briefly free to move. In the absence of the magnetic field, they would simply disperse randomly, but in the presence of the field they line up with the lines of force.

Similarly with sparse constructive node irradiation, the tapping of the sheet is represented by the absorption and release of photons, while the EM field of the wavelength of the laser frequency represents the overall magnetic lines of force.

Through this process the molecule is subjected to the very long wavelength field effect of the EM wave at a given polarization that tends to pull the molecule in line with the field at the same time as one or more atoms in the molecule absorb individual photons from the node. This ensures that the molecule will ring at its natural frequency and will tend to orient with the field.

When more specific effects are desired than the simple “bang and ring” of the low frequency flatten and stretch effect, a specific bond resonance frequency for a given molecule would be applied. This would be at a much higher frequency and would cause very specific alterations in the molecule rather than the general homogenization effect caused by lower frequencies and sparser nodes. These alterations range from breaking specific molecular bonds, perhaps with a view to immediate incorporation of the cleavage fragment into another molecule, to causing a particular end of a molecule to preferentially bond in a polymerization process.

The estimated dosage of laser irradiation to achieve the general molecular homogenization effect has been estimated for the betaine molecule to be as rapid as 3 seconds per mole per milliwatt of applied laser energy under ideal conditions of particle exposure. Smaller particle size and dispersion or air suspension of particles will tend to make the process more efficient. Using the molar ratio of treatment for betaine gives an approximate fastest rate of homogenization effect at a dosage of 30 seconds per kilogram per milliwatt.

For larger molecules the duration of treatment per mole per milliwatt will be greater, but this will roughly increase in proportion to molecular weight so that the fastest effective treatment duration per kilogram will remain roughly the same. Treatment duration longer than required will not tend to further increase the effect nor will it likely degrade to purely thermal effects as long as the radiation applied is generally below or much below that which would raise the bulk temperature of the treated species by more than one degree Celsius per mole per second.

For practical purposes, to increase the tendency to maximum homogenization effect, treatment dosage has usually ranged from 0.03 to 0.05 kilograms per minute per milliwatt of sparse constructive node laser irradiation.

Comparing and contrasting the sparse constructive node laser EM irradiation to routinely configured continuous wave lasers, several essential differences appear. As noted in Strachan PCT/GB00/03280, the depth of penetration of visible wavelengths of conventional laser EM through an intensely scattering medium such as human skin is typically less than 5 mm even at the most penetrating wavelengths. In contrast, the pulse train of sparse constructive nodes, by virtue of much decreased scattering, may have effective coherent penetration of 60 mm through skin and even greater penetration through other tissues.

For combined modality treatments such as photodynamic therapy (PDT) which combines the effects of photosensitizing compounds such as benzoporphyrin derivative with the application of photons to cause photo-oxidation reactions resulting in elimination of pathologic tissue, application of sparse constructive node laser irradiation could greatly extend the current reach of this treatment modality.

For practical purposes, for example in the treatment of malignancy, photodynamic therapy is limited to application to endoscopically visible lesions in the respiratory or digestive tracts, or to other areas topical to the skin or directly accessible with a fiberoptically transmitted laser EM signal. Greater depth of effective coherent signal penetration could make a wider range of malignant lesions and other PDT sensitive conditions accessible to treatment with this generally effective and well tolerated modality.

Conventional lasers usually produce equal ratios of constructive and destructive nodes, representing a small ratio of overall output energy, generally much less than 1% of output energy. Even a modulated beam of conventional laser EM will not have a sufficient duration of relaxation time before the entry of the next photon to permit efficient per cycle release of absorbed photons. In contrast, in a sparse constructive node beam, destructive nodes are highly dominant and highly structured in the emission from the optical device.

When an atom absorbs a photon from a conventional laser beam, the atom becomes highly reflective of additional photons, until the absorbed photon is emitted. Because of the absence of sufficient destructive nodes to permit enough conduction of energy away from the atom before the next packet arrives for absorption, the atom tends to remain in an excited and reflective state.

In contrast, with sparse constructive node laser EM irradiation, each atom is excited at resonance with the molecular backbone. The intervening traverse of the destructive node permits the collapse to ground state with photon emission. The atom is then ready to absorb the next photon from the constructive node when it arrives, to sustain and magnify the resonance effects.

The high level of surface reflectance due to molecular absorption of photons from conventional laser irradiation causes intense photon scattering at the surface irradiated. All light scatters and plumes at the beginning of the entry of the beam through the absorptive medium. There is a flare at the surface only, with scattering thereafter, preventing potential resonance effects.

In contrast, in a sparse constructive node beam, the constructive nodes are rare and destructive nodes dominate. The space between the constructive nodes allows intramolecular resonance and intermolecular tuning. Unlike a bright continuous wave beam, the sparse constructive node effect is spread through the medium, so the effect is less scattering, permitting deeper penetration and greater degrees of molecular resonant stimulation.

Although a pulsed conventional laser with a short pulse duration time may overcome some of the limitations of continuous wave lasers for resonant stimulation, the absence of predominant destructive nodes in the pulse waves will still tend to result in high degrees of surface scattering. The ultrashort constructive nodes with the relaxation phase of the destructive nodes will tend to enhance the performance of a sparse constructive node beam over an ordinary pulsed laser beam for acoustic resonance and coherent depth of penetration.

The performance of a conventional pulsed laser in stimulating molecular resonance would be expected to improve if the pulses themselves were configured into sparse constructive nodes by passing the pulsed beam through a Strachan interference optical device.

Conventional continuous wave laser irradiation has a high probability of hitting a molecule more than once per resonant cycle of the irradiated molecule. In contrast, with sparse constructive node laser irradiation, the probability of an atom being hit by another photon in a period of excitation is low, but once per cycle is high.

By analogy, consider several bells lined up in a row. The deluge of scattering photons in a continuous wave laser beam squashes the resonance of the first bell. In comparison, sparse constructive node irradiation stimulates resonant ringing of the first bell that then stimulates the other bells to begin to ring, translating the resonant signal deeper into the irradiated medium. In this manner the sparse constructive node can stimulate intramolecular and intermolecular resonance. In the sparse constructive node, the probability of photon arrival at the intended molecule is adjusted to permit building cycles of molecular resonance that increase molecular kinetic energy.

Continuous wave laser irradiation causes an abrupt increase of thermal energy at the absorptive surface that is conducted randomly from the locus of stimulation. In contrast, sparse constructive nodes deliver lower total energy, but deliver this energy through resonance to very specific locations. The structured energy retained per molecule can be many times greater than that delivered through conventional continuous wave laser stimulation, increasing the reactivity of the treated molecules.

Continuous wave laser irradiation excites molecules at the time there should be a trough. This is akin to kicking a swing continuously, which will deliver impulses out of phase with the natural frequency of the swing cycle. Sparse constructive nodes deliver less energy, but provide it in phase with the natural frequency of the molecule stimulated. The kinetic energy that builds in the backbone structure of the molecule will tend to stretch and flatten the molecule.

In addition, this will tend to remove bonded water from the molecule, resulting in a dryer structure, even in a molecule already in dry powder form. Hydrogen bonds may be rearranged, altering solubility factors and potentially modifying the free energy of chemical bonds thus restructured.

In general, the stretched and flattened shape stimulated by sparse constructive node laser irradiation will tend to be highly homogeneous from molecule to molecule. The molecules thus homogenized will tend to have a lower overall energy configuration with a higher electric and magnetic field moment than molecules not homogenized with this process.

Homogeneity, flattened and stretched shape, and high electric and magnetic field moments favor efficient binding of substrates to enzymes or ligands to receptor sites, especially the binding of the next reactant molecule to an enzyme if it is highly similar in shape to the reactant that has just been released from the enzyme.

Conventional continuous wave laser irradiation has a low probability of maintaining resonance, exciting everything and delivering photons at the wrong time. It is therefore inefficient at changing the shape of molecules in a consistent fashion. In contrast, sparse constructive node laser stimulation is intrinsically efficient at stimulating the natural frequencies of molecules and homogenizing their shape.

Chemical and especially enzymatically catalyzed reactions are highly shape dependent. The relatively random effects on molecular shape caused by conventional laser irradiation may do little to increase efficiency of chemical reactions other than rate acceleration due to thermal heating alone (with the exception of wavelength specific photochemical reactions). In contrast, sparse constructive node laser irradiation can provide vastly greater control over chemical reactions. This can be through homogenization of substrate or specifically heating a bond that is desired to be more active in the reaction process.

Sparse constructive node stimulation is especially advantageous in a reaction in which heating would damage one reagent while leaving the other unharmed. Sparse constructive node irradiation can be used to heat the temperature resistant substrate while leaving the temperature sensitive reactant unharmed.

For a chemical reaction, especially an enzyme moderated one, the homogenization process can increase the chemical potential, or potential difference, that drives it. The chemical potential depends on the intrinsic properties of the substrate and product molecules, and their concentrations. If a reaction process A+B=C+D is reversible, the direction and rate of the reaction depends on the properties of A, B, C, and D and the effective quantities of each. The more A and B one adds, the more C and D are formed, and vice versa; also, if C or D is removed as fast as it is made, the reaction is driven to the right.

Homogeneity in the reactants is equivalent to increased concentration because the reaction surface of the cell can be more regular and thus more compact, and because enzymes will bond considerably faster to a molecule identical to that just released than to one even slightly dimensionally different.

Considering the chemical potential as defined above with respect to enzyme moderated reactions, it can be seen that increasing the homogeneity of one or more of the reactants is equivalent to both increasing the effective concentration of the reactant and lowering the first stage energy of binding the reactant to the enzyme, because the enzyme that fits one molecule needs virtually no energy to fit the next if the substrate shapes are identical, let alone requiring, as in the case of highly inhomogeneously crystallized reactants, the manufacture of a wider range of enzymes to moderate a given reaction. The energy potential of the reactions rise because of the increase in effective concentration of the reactants.

Some molecules will have a change in the free energy of certain bonds due to the overall shape change of the molecule. Depending on the product desired, this may help or hinder the production of a given product, whereas increasing the similarity molecule to molecule of bond energy and dimension will always facilitate the production of a product in an enzyme moderated reaction. The effect on each interaction may be tiny but the overall effect can be substantial.

The rate at which a reactant can be supplied to an enzyme or receptor is directly proportional to the self-similarity of the molecules of the reactant or the receptor ligand. Thus a given quantity of reactant or receptor ligand can generate more product or stimulate more potent receptor effects the greater the self-similarity of the reactant molecules to each other. Molecules irradiated with sparse constructive nodes will in general be highly similar to one another in terms of shape and dimensions, distribution and location of water, and the presence of relatively high electric and magnetic field moments for that molecular species.

One particular advantage of this invention is that the homogenizing action on a dry powder of L-arginine can be translated into differential effects in vitro after dissolving the dry powder into solution. Thus, the treatment of the dry power causes a structural change in the molecules that changes the bioavailability and/or the physiologic reaction to the substance. This, in turn, materially alters the utilization of the substance by a body—and in particular, a mammalian body. The substance is sufficiently stable to maintain the effects of molecular stimulation that creates an enhanced biological effect even after the substance is dissolved in solution. In addition, the use of the method on a solution containing the substance can produce similar enhancements.

In accordance with another aspect of the present invention, the method is used to modify the physiologic production of nitric oxide from the amino acid L-arginine. For a given molar concentration of L-arginine, depending on the laser resonance applied, the production of nitric oxide from macrophages in vitro may be statistically significantly increased or decreased. Thus, by utilizing the present invention, one can increase desirable byproducts or decrease undesirable byproducts associated with a nutrient, pharmacological agent, or other bioactive substance in the body.

In accordance with another aspect of the present invention, the method involves increasing the potency of L-arginine to amplify a wide range of reported physiologic benefits of arginine-derived nitric oxide (ADNO). These include, but are not limited to, the effects of ADNO to lower blood pressure with a minimum of physiologic side effects; to dilate bronchial tubes and improve pulmonary function test results; to mediate long-term potentiation in neural tissue and thereby promote memory function; to improve oxygen delivery in tissues through hemoglobin-related mechanisms; to reduce LDL and total cholesterol levels and LDL oxidation; to promote the release of growth hormone and its wide range of anti-aging benefits; to improve microvascular blood flow and tissue perfusion; to increase the immunologic actions of ADNO that include generation of nitric oxide “bullets” for direct anti-microbial and anti-tumor effects, increased natural killer cell activity, and enhanced cytokine production, e.g., tumor necrosis factor-alpha.

In addition, the ADNO effects mediated through increased cyclic guanosine monophosphate (cyclic-GMP) generation can also be enhanced; these include the effects of ADNO via cyclic GMP to enhance male sexual potency, and probably female vaginal lubrication, as well as increased genital sensitivity in both men and women.

In accordance with still yet another aspect of the invention, it has been found that reducing the potency of L-arginine for ADNO production may preserve nutritive benefits of L-arginine while reducing the risk of adverse effects of L-arginine supplementation that can occur in selected circumstances in susceptible individuals. These situations include but are not limited to persons with Herpes simplex viral infection that may have an increased risk of outbreaks with L-arginine supplementation and persons with inflammatory conditions for whom supplemental L-arginine may aggravate nonspecific inflammatory symptoms. In particular, reduced risks of Herpes simplex outbreaks may result from the use of reduced potency L-arginine in conjunction to the addition of at least one gram daily of the amino acid L-lysine.

An additional aspect of the present invention involves the ability to modify hydrophobic and hydrophilic interactions through laser resonance, as observed through X-ray crystallography. In particular, the method can be used to develop new forms of L-arginine hydrochloride and other molecular structures in both dry states and in solution.

For example, the disclosed method was used to compare the crystal structures of L-arginine hydrochloride grown without versus with laser resonant stimulation. L-arginine hydrochloride was dissolved in de-ionized water and then crystallized without and with laser stimulation by slow evaporation at room temperature. The control L-arginine hydrochloride upon crystal structure solution was found to have the typical features of L-arginine hydrochloride monohydrate reported in the literature, with one molecule of water per molecule of L-arginine hydrochloride in the crystal lattice. The laser treated L-arginine hydrochloride demonstrated a significantly different crystal structure, an L-arginine hydrochloride without water in the crystal lattice with different unit cell characteristics, and a very high level of uniformity of elongation of the nitrogenous side chain.

The sparse constructive node laser treated L-arginine hydrochloride showed the predicted effects of high levels of homogenization and reduction of the bonded water in the molecular structure. This result suggests the ability to modify a wide range of molecular structures in intended ways, both in the dry state and in solution.

In accordance with one aspect of the present invention, the method of Strachan (or other molecular modification methods) can be used to modify the immunologic effects of a blend of a complete spectrum of amino acids.

In accordance with this aspect of the invention, a highly immunostimulant amino acid or blend of amino acids is laser treated. The laser modifies the structure of the amino acid(s) to reduce the immune stimulation to the baseline level without the amino acids. In other words, modifying the amino acid structure reduces negative immune reactions to the amino acids. Such a modified form of nutrition may be highly desirable for persons with poor nitrogen balance and immune overactivity, e.g., autoimmune diseases, food allergies, and other inflammatory conditions such as inflammatory bowel disease. Thus, in accordance with this aspect of the invention a route of elemental, readily absorbed and assimilated nutrition is provided that will not further aggravate an underlying inflammatory condition.

In accordance with still yet another aspect of the present invention, there is disclosed an improved method of administration of dietary nucleic acid elements and dietary nucleotide precursors. In a presently preferred embodiment of one aspect of the invention, a method is disclosed that does not require parenteral administration, yet provides better delivery of nucleic acid elements to tissues than oral ingestion.

Metabolic incorporation studies indicate that orally administered purines and pyrimidines undergo significant metabolic degradation both by intestinal bacteria and the intestinal epithelium. Orally administered pyrimidines show an incorporation level of approximately 5% in the intestinal lining and only 3% in the liver. Orally ingested purines are even more extensively oxidized such that less than 1% of purine nucleosides are incorporated into hepatic nucleic acid pools.

Studies with radiolabeled purines show that intravenous injection compared to oral ingestion results in vastly higher incorporation levels in certain metabolically active tissues, with IV:oral incorporation levels as high as 29-59:1 in pituitary, thymus, salivary, thyroid, adrenal, and lymphoid tissues.

Recent evidence indicates that although the body can manufacture nucleic acid bases from amino acids and other precursors, some tissues have a synthetic capacity below that required for optimum tissue maintenance, repair, and regeneration. This may be particularly true of lymphoid tissues, especially under conditions of stress. Numerous studies have shown marked immunologic benefits of supplemental nucleic acid elements especially on improved cellular immunity. Animal studies have shown significant improvements in outcomes for systemic bacterial and fungal infections, as well as malignancies. Human studies show marked improvement in cellular immunity as well as enhanced intestinal growth, maturation, and repair.

To overcome the limits of oral ingestion, this disclosure presents, as a preferred embodiment of one aspect of the invention, the delivery of nucleic acid elements via an intra-oral spray formula or by rectal or vaginal suppository. Absorption studies suggest that nutrients applied to the oral mucosa may achieve up to 90% direct systemic absorption while also overcoming the limitations of hepatic first pass metabolism. These elements may include one or more of the following forms: laser treated DNA and RNA nucleobases, nucleosides and deoxynucleosides, and nucleotide and deoxynucleotide monophosphates, diphosphates, and triphosphates. It is possible that laser treatment of nucleotides and deoxynucleotides may at least temporarily generate higher energy more highly bioactive high energy phosphate groups.

This formula may also contain one or more laser homogenized amino acids, in particular those amino acids known to be precursors of endogenous nucleobase synthesis: glycine, L-glutamine, L-serine, and L-aspartic acid. This formula may also contain one or more laser treated vitamins, minerals, trace elements, and other nutrient cofactors that support nucleotide metabolism. This laser irradiated formulation for enhanced nucleic acid metabolism may also be provided intravenously or through other parenteral injection routes, such as subcutaneously or intramuscularly. Although improved absorption through oral ingestion of laser treated versus untreated nucleic acid elements is anticipated, significant intestinal mucosal degradation remains likely.

In accordance with yet another aspect of the present invention, the method is used to create a homogenized form of trimethylglycine (TMG). TMG, also known as betaine, is a methyl group donor that participates in many fundamental chemical pathways in the body.

TMG is derived from the simplest of the amino acids, glycine, that has 3 methyl groups replacing the 3 hydrogen atoms of the amino group. X-ray crystallography comparing the control versus laser treated hydrochloride of betaine shows the predicted effects of molecular homogenization and the flattening and stretching of molecular shape.

Homogenization, creating greater self-similarity of molecular shape, is shown by a significant reduction of crystal defects in the laser treated sample compared to the control sample, despite both samples being crystallized by slow evaporation at room temperature. Increased defects in the control crystal would be predicted from the greater range of shapes of the untreated compound having difficulty fitting uniformly in the crystal lattice.

In contrast, consistent flattening and stretching of shape from molecule to molecule permits more rapid incorporation into a uniform crystal lattice. The X-ray crystallographic analysis shows the explicit 3-dimensional shapes for the control and treated hydrochlorides of betaine, and is consistent with the predicted changes in shape.

The laser treated sample in particular shows flattening and stretching of the carbon-nitrogen bonds of the amino methyl groups, and to a lesser degree also suggests flattening and stretching of the carbon-hydrogen bonds of the methyl groups, as well as the carbon-oxygen bonds of the carboxyl group. This flattened shape will tend to have higher field energy with reduced bond energy, favoring lower energy of enzymatic binding and higher enzymatic reactivity.

It is understood from the current evidence, that this activated state creates a reactive methyl group that facilitates a variety of biological processes in the body, and provides numerous benefits to the body.

For example, it has been found that betaine can reduce blood levels of homocysteine, a substance that has been linked to numerous negative physiological conditions, through the enzyme betaine-homocysteine methyltransferase that transfers a methyl group from betaine to homocysteine to convert it to the amino acid methionine.

By providing the activated betaine combined with nutrients that serve as cofactors in the methyl group transfer pathways in the body, significant homocysteine reductions can be achieved, thereby limiting the risk of heart attack, strokes, dementia, pre-eclampsia, and certain malignancies, especially of epithelial origin, such as cervical, colon, and possibly bronchogenic neoplasms.

The activated betaine and cofactors can also be used to reduce anxiety, depression, hostility, paranoia, somatization (body aches and pains), and obsessive-compulsive symptom scales.

In light of the present disclosure, it will be appreciated that a variety of chemicals can be modified in accordance with the principles of the present invention. In particular, any organic molecule whose shape may be twisted or deformed through the processes of chemical synthesis, purification, or drying may be homogenized to a more self-similar and more bioavailable shape configuration.

This process will tend to be relatively less efficient for small molecules with few degrees of rotational freedom or planar cyclic molecules; whereas molecules with long unsaturated mobile side chains, such as L-arginine, that may take numerous ground state configurations, are well suited to homogenization and reshaping through this process.

The enhanced amino acids and other substances described in this invention may be provided as dry powders or as solutions through several routes of administration. These include oral spray, mucosal, oral ingestion, enteral feeding tube, parenterally through various routes, and topically.

### BRIEF DESCRIPTION OF THE DRAWINGS

The above and other objects, features and advantages of the invention will become apparent from a consideration of the following detailed description presented in connection with the accompanying drawings in which:

FIG. 1A shows the X-ray powder diffraction (XRPD) pattern of control simvastatin sample Sim 1A.

FIG. 1B shows the XRPD pattern of laser treated simvastatin sample Sim 1B demonstrating increased crystallinity.

FIG. 2A shows the XRPD pattern of laser treated simvastatin sample Sim 2A demonstrating lower intensity reflections indicative of amorphous content.

FIG. 2B shows the XRPD pattern of laser treated simvastatin sample Sim 2B demonstrating very low intensity reflections indicative of an even higher amorphous content.

FIG. 3A shows frontal and lateral photomicrographs of crystals of control untreated L-arginine hydrochloride monohydrate.

FIG. 3B shows frontal and lateral photomicrographs of crystals of laser treated anhydrous L-arginine hydrochloride.

FIG. 3C shows X-ray crystallographic results of laser treated or modified L-arginine hydrochloride.

FIG. 4A shows a quantitative EEG (QEEG) study of baseline alpha brainwave coherence;

FIG. 4B shows a QEEG study of alpha brainwave coherence one hour after ingestion of untreated amino acids;

FIG. 4C shows a QEEG study of alpha brainwave coherence one hour after ingestion of laser treated or modified amino acids;

FIG. 5A shows lateral photomicrographs of crystals of control betaine hydrochloride and laser treated or modified betaine hydrochloride;

FIG. 5B shows frontal photomicrographs of crystals of control betaine hydrochloride and laser treated or modified betaine hydrochloride;

FIG. 5C shows x-ray crystallographic results of intermolecular hydrogen bonding for control betaine hydrochloride;

FIG. 5D shows x-ray crystallographic results of intermolecular hydrogen bonding for laser treated or modified betaine hydrochloride;

FIG. 5E shows x-ray crystallographic results for the molecular structure of control betaine hydrochloride as dashed lines and for laser treated or modified betaine hydrochloride as solid lines, showing backbone models in the upper diagram and ball and stick models in the lower diagram;

FIG. 6A shows a diagram of the methyl group transfer metabolic pathways;

FIG. 6B shows a graph demonstrating reduced homocysteine levels after treatment with modified betaine;

FIG. 6C shows a graph demonstrating a control group;

FIG. 6D shows a graph demonstrating reduced homocysteine as a function of treatment quantity in a subgroup of subjects with moderately elevated baseline homocysteine levels (≧10);

FIG. 6E shows a graph demonstrating reduced anxiety as function of treatment quantity;

FIG. 6F shows a graph demonstrating reduced somatization as a function of treatment quantity;

FIG. 6G shows a graph demonstrating reduced obsessive compulsive symptoms as a function of treatment quantity; and

FIG. 6H shows a graph demonstrating reduced depression as a function of treatment quantity;

FIG. 61 shows a graph demonstrating reduced paranoia as a function of treatment quantity;

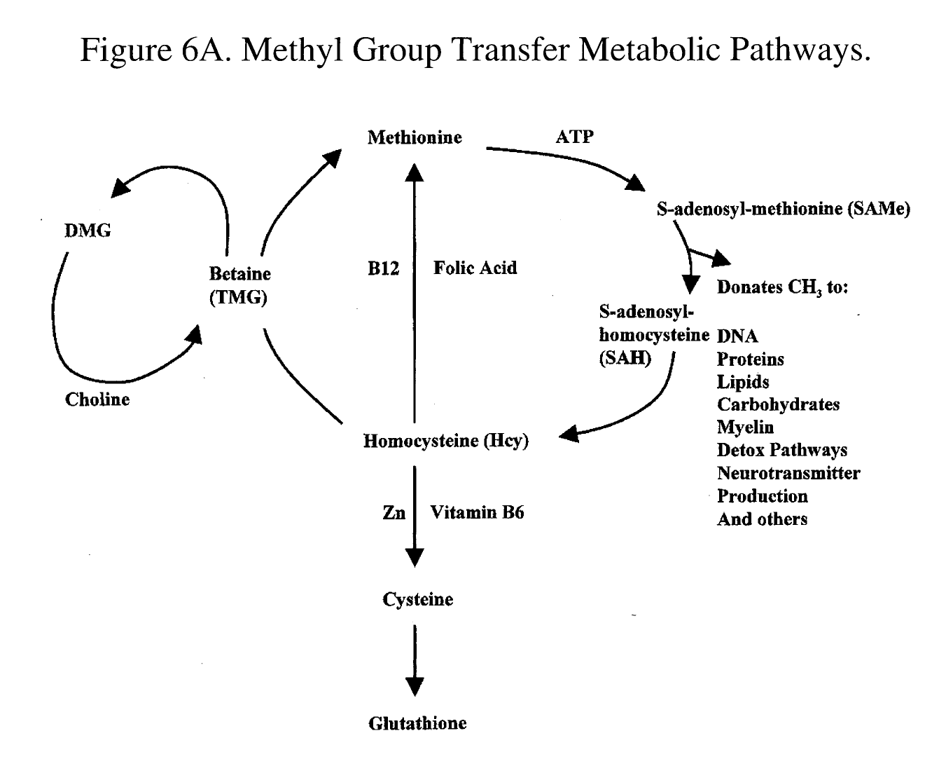

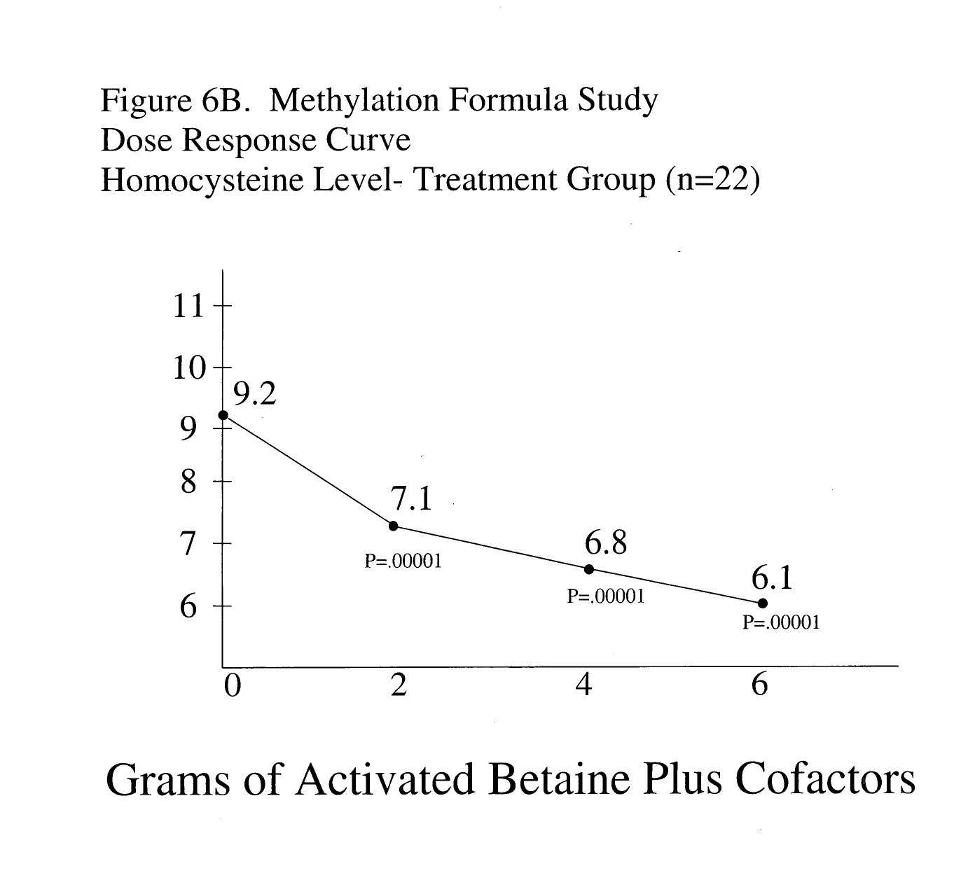

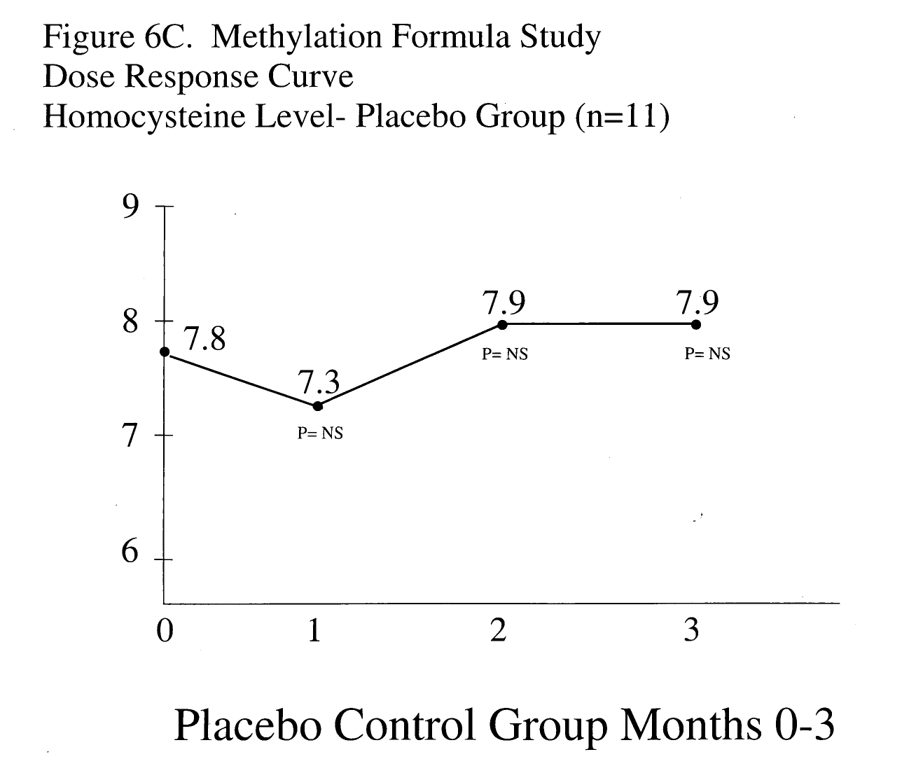

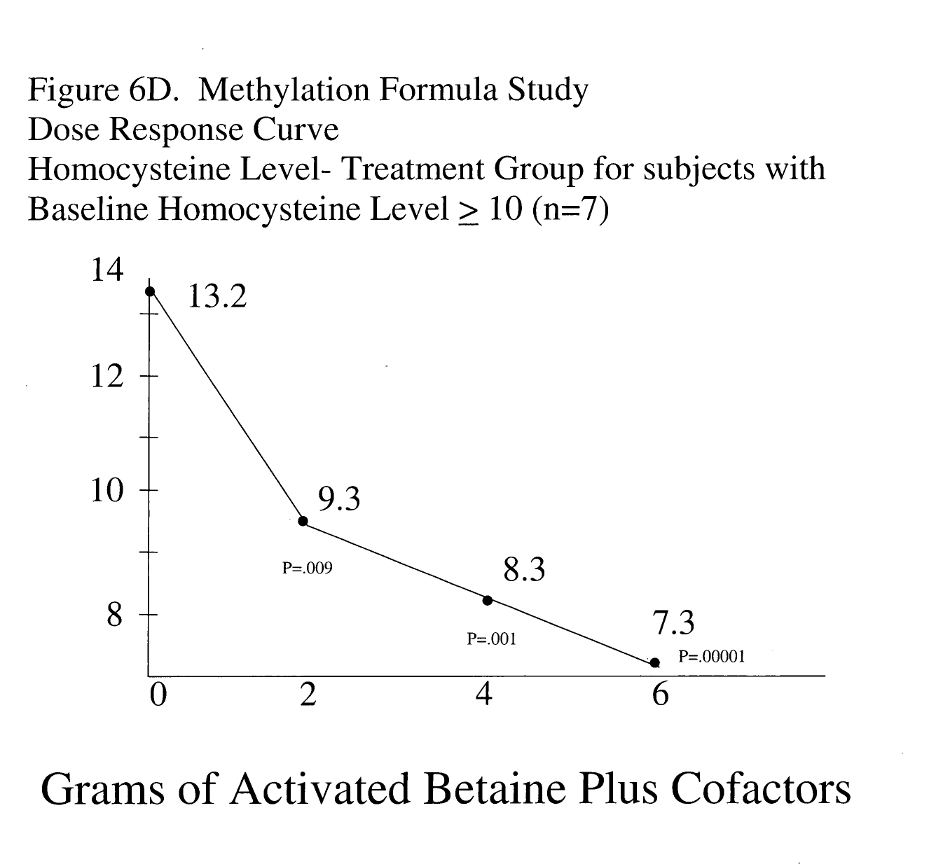

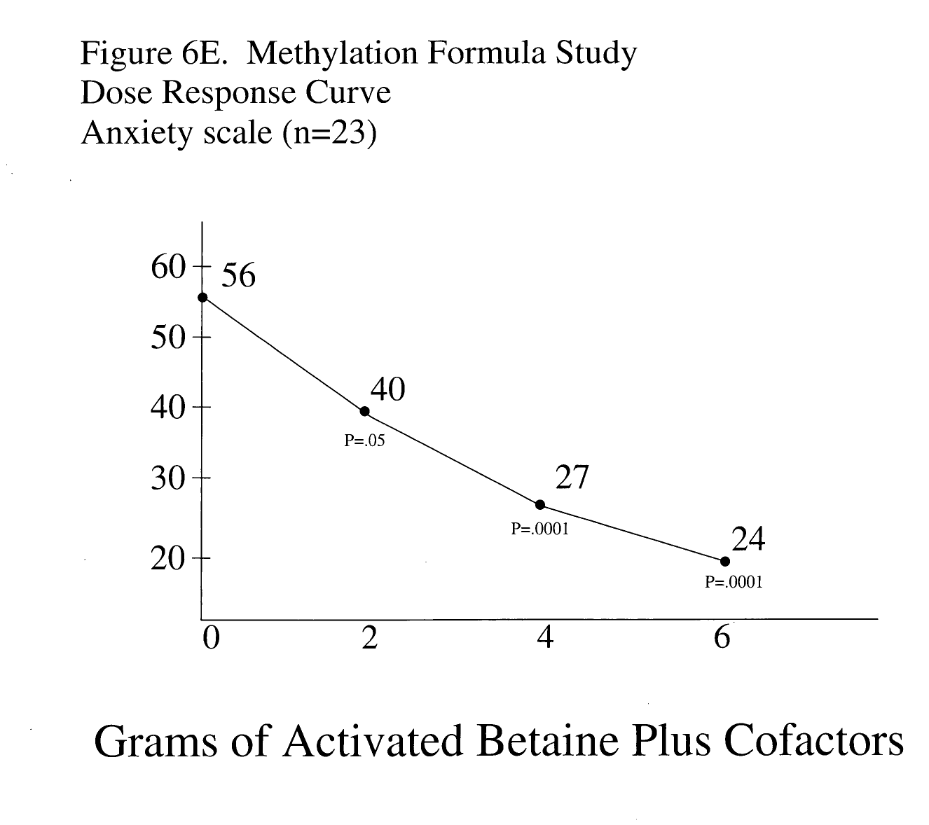

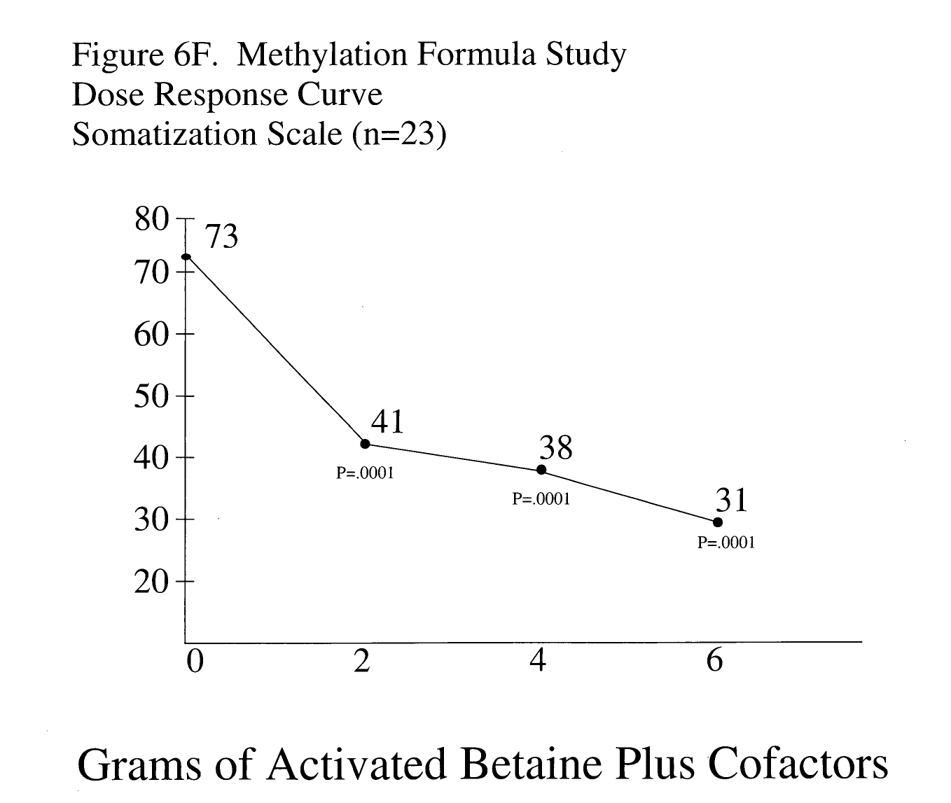

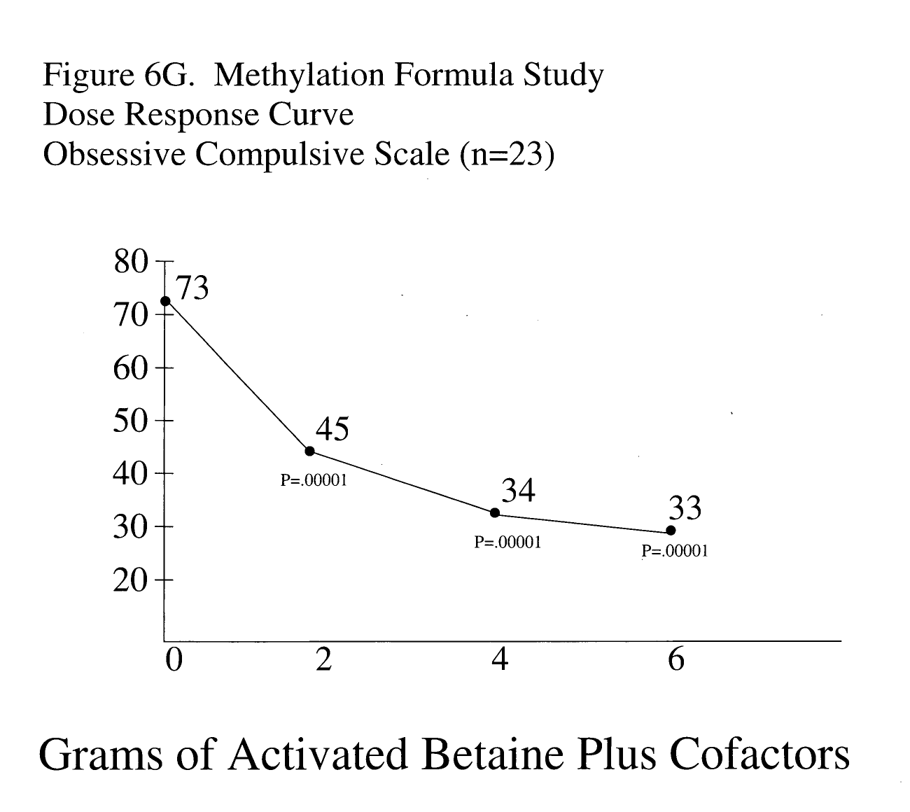

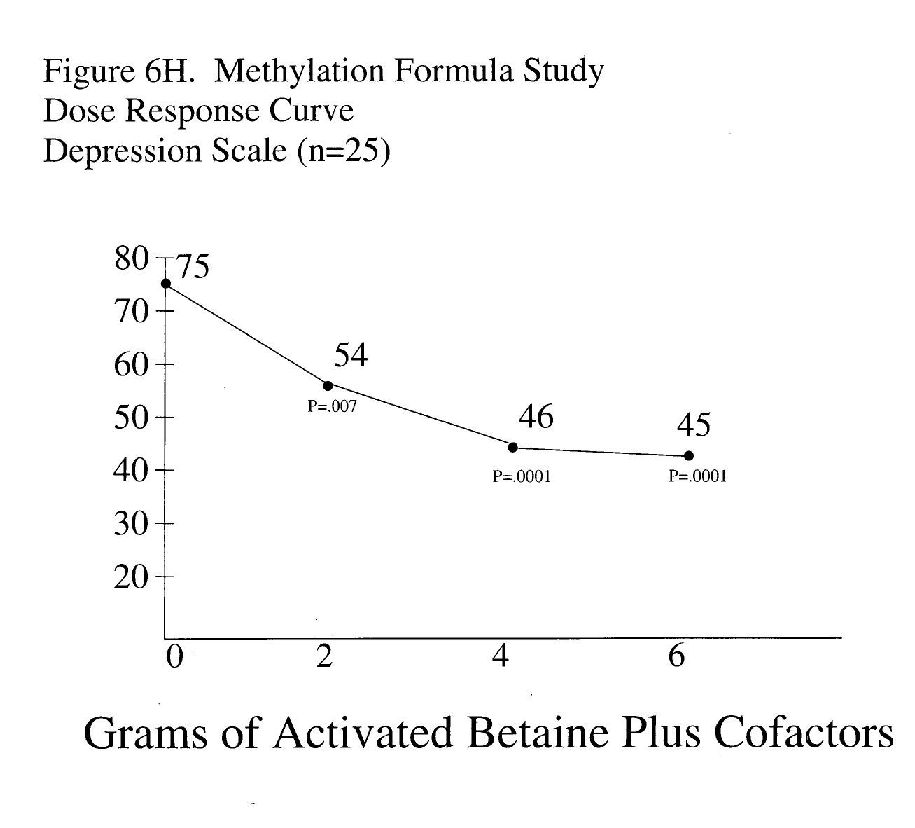

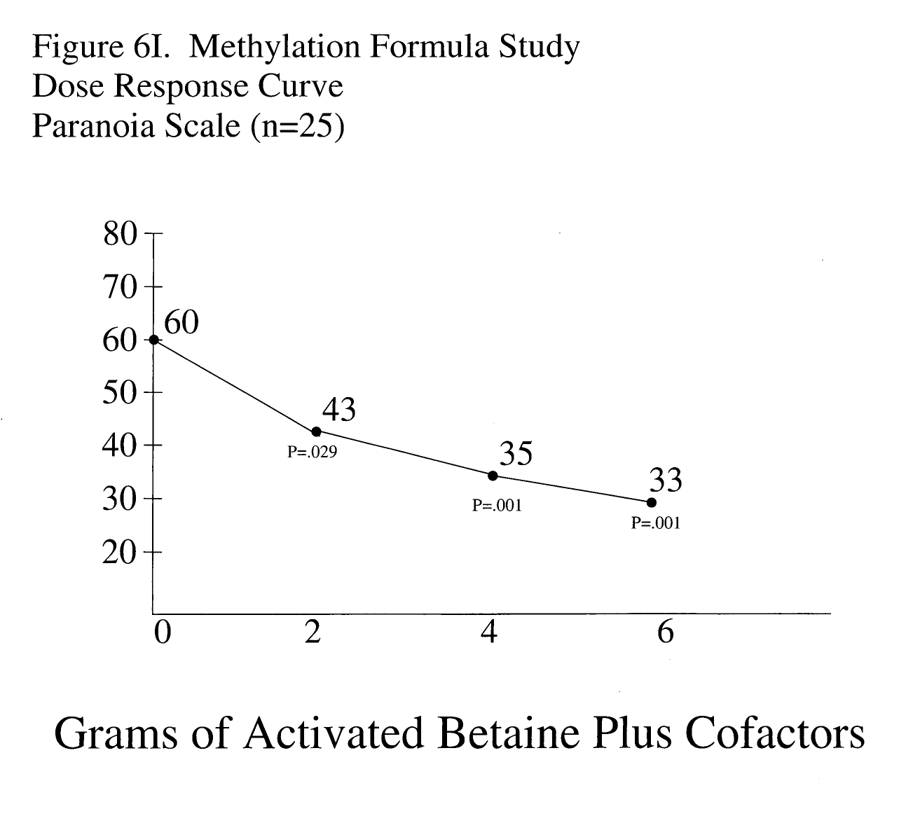

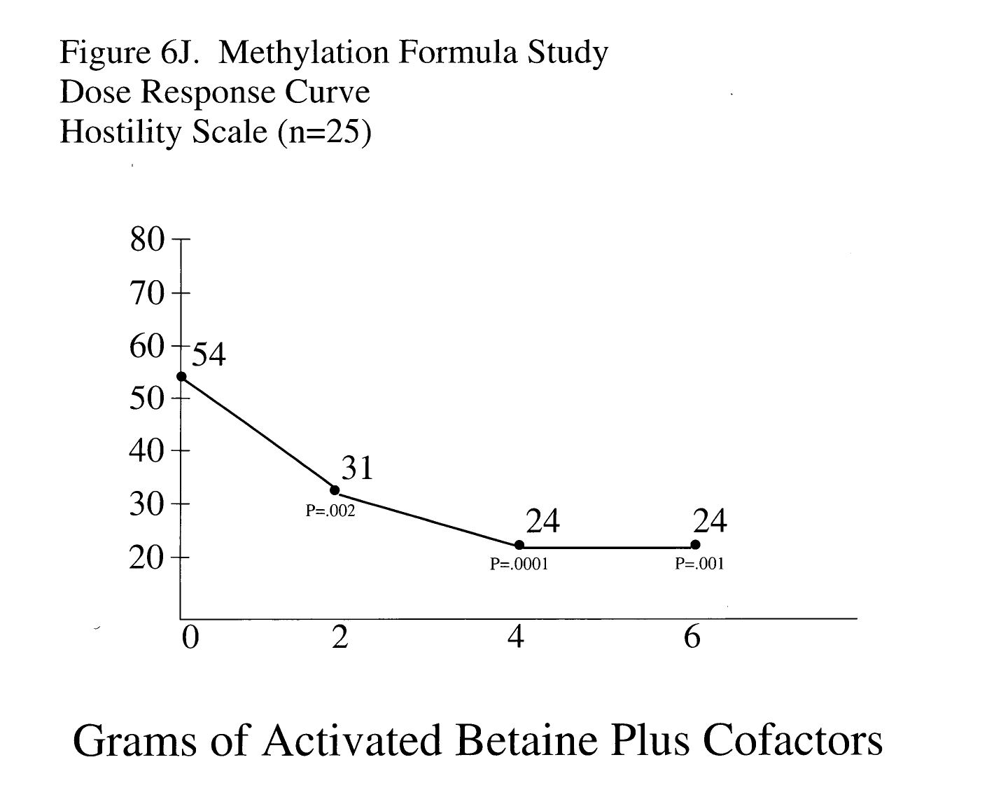

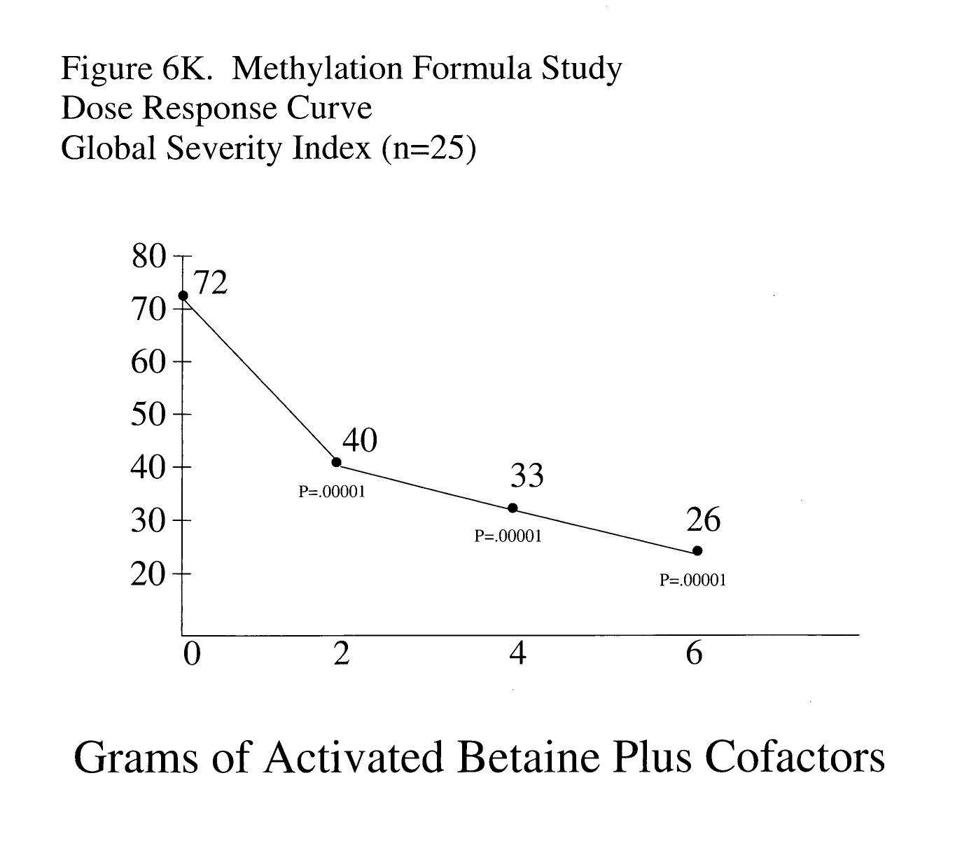

FIG. 6J shows a graph demonstrating reduced hostility as a function of treatment quantity;

FIG. 6K shows a graph demonstrating reduced global severity index as a function of treatment quantity.

### DETAILED DESCRIPTION OF THE INVENTION

The various aspects of the present invention will now be discussed so as to enable one skilled in the art to make and use the invention. It is to be understood that the following descriptions are only exemplary of the principles of the various aspects of the present invention, and should not be viewed as narrowing the pending claims. It is also to be understood that each embodiment may not accomplish each object of the invention, but provides one or more advantages over the prior art.

### EXAMPLE 1

### Production of Highly Homogeneous Simvastatin with Increased Crystallinity through Applying Laser Acoustic Resonance

Two Samples of United States Pharmacopeia (USP) reference standard simvastatin of 21 mg each were used for this study. Each sample was dissolved in 200 mg of 100% ethanol and placed in a 10×35 mm polystyrene Petri dish. Sim 1A was prepared as the untreated control and Sim 1B was prepared for treatment with modulated sparse constructive node laser acoustic resonance to assess for differences in crystallinity using X-ray powder diffraction.

Both samples were crystallized by slow evaporation at room temperature. Sim 1A served as the control and had no additional treatment modalities applied to it. Sim 1B was treated with a 670 nm diode laser of 4.7 mW primary power phase conjugated through the optical elements to a power level of 2.35 mW. The beam was modulated at 10 MHz and passed through the middle of the fluid meniscus of the solution until Sample 2 was fully crystallized. Both samples were then sent to a reference lab for X-ray powder diffraction (XRPD) studies.

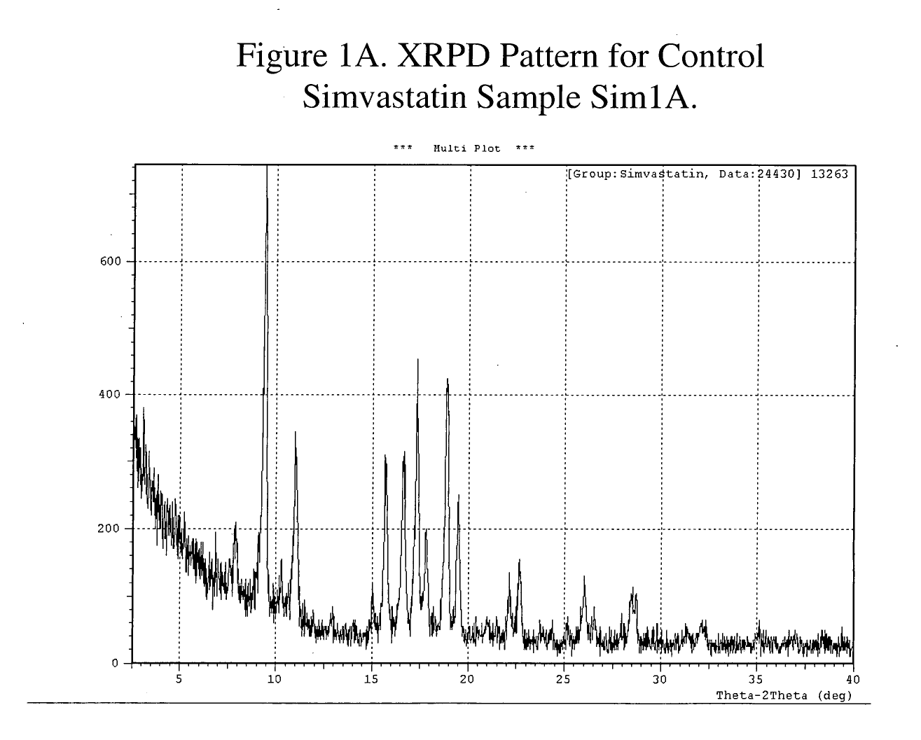

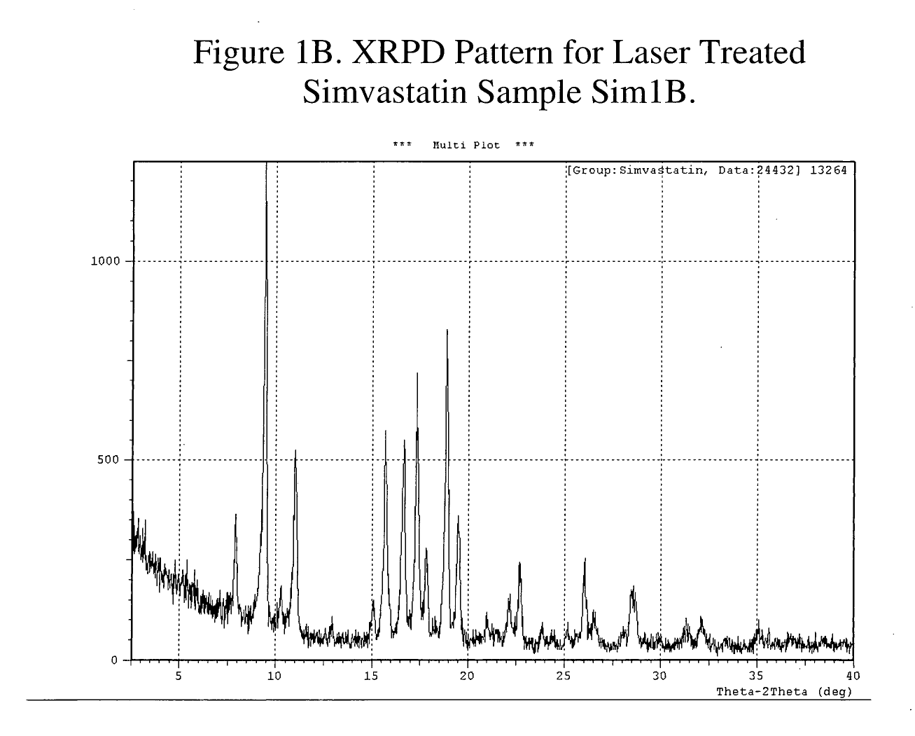

FIG. 1A shows the XRPD pattern for Sim 1A, the control reference standard of simvastatin. FIG. 1B shows the XRPD pattern for laser acoustic resonance treated Sim 1B. The corresponding peaks in FIG. 1B are ˜70% greater in amplitude than FIG. 1A. The sharper resolution and significantly increased amplitude of the reflections for Sim 1B indicate a higher degree of crystallinity for Sim 1B.

Based on thermodynamic considerations, increased crystallinity is associated with increased stability of the crystal form. For storage purposes of a pharmaceutical or other compound, a more highly crystalline form is more likely to maintain its form and characteristics for a longer period of time, and thus will tend to have a significantly longer shelf life.

Perhaps more importantly, the risk of converting to a different crystal form during storage may be decreased, as such conversions can greatly alter the effects of the compound in the body. Particularly for metastable crystal forms that are not already in the lowest free energy form, increasing the crystallinity of the metastable form may reduce the risk of the very undesirable conversion to the more stable form that has usually been avoided because of poor solubility and low bioavailability. Increasing the probability of maintaining the metastable form in a predictable way can provide a great advantage for those compounds that must be provided in this form to be sufficiently soluble and bioavailable to be of clinical benefit.

### EXAMPLE 2

### Production of Partially Amorphous Simvastatin Through Application of Laser Acoustic Resonance

Two Samples of United States Pharmacopeia (USP) reference standard simvastatin of 21 mg each were used as an extension of the study described in Example 1. Each sample was dissolved in 200 mg of 100% ethanol and placed in a 10×35 mm polystyrene Petri dish. Sim 2A and Sim 2B were prepared for treatment with modulated sparse constructive node laser acoustic resonance to assess for differences in crystallinity using X-ray powder diffraction.

Both samples were crystallized by slow evaporation at room temperature. Sim 2A was treated with a 458 nm pumped argon gas laser of 2.1 mW primary power phase conjugated through the optical elements to a power level of 1.05 mW. The beam was modulated at 6.4 MHz and passed through the middle of the fluid meniscus of the solution until Sample 2A was fully crystallized. Sim 2B was treated with a Quantel Nd-YAG pulsed laser at 467 nm with an average pulse amplitude of 2-5 mJ/pulse over 5 nanoseconds, 12 pulses per second. The optics were adjusted to maximum cancellation and the beam was passed through the middle of the fluid meniscus of the solution until Sample 2B was fully crystallized. Both samples were then sent to a reference lab for X-ray powder diffraction (XRPD) studies.

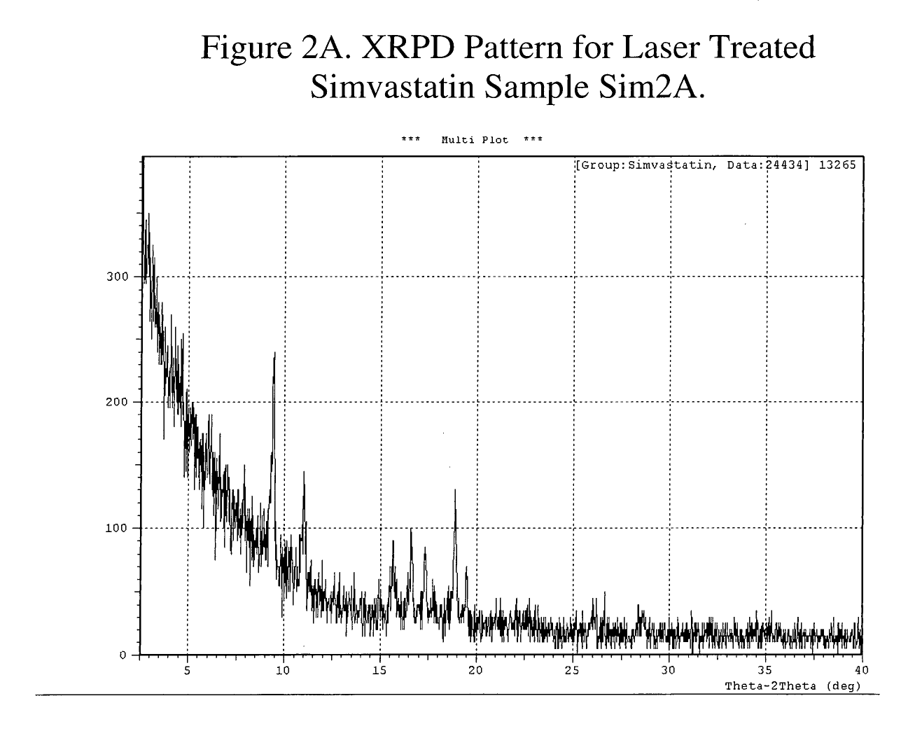

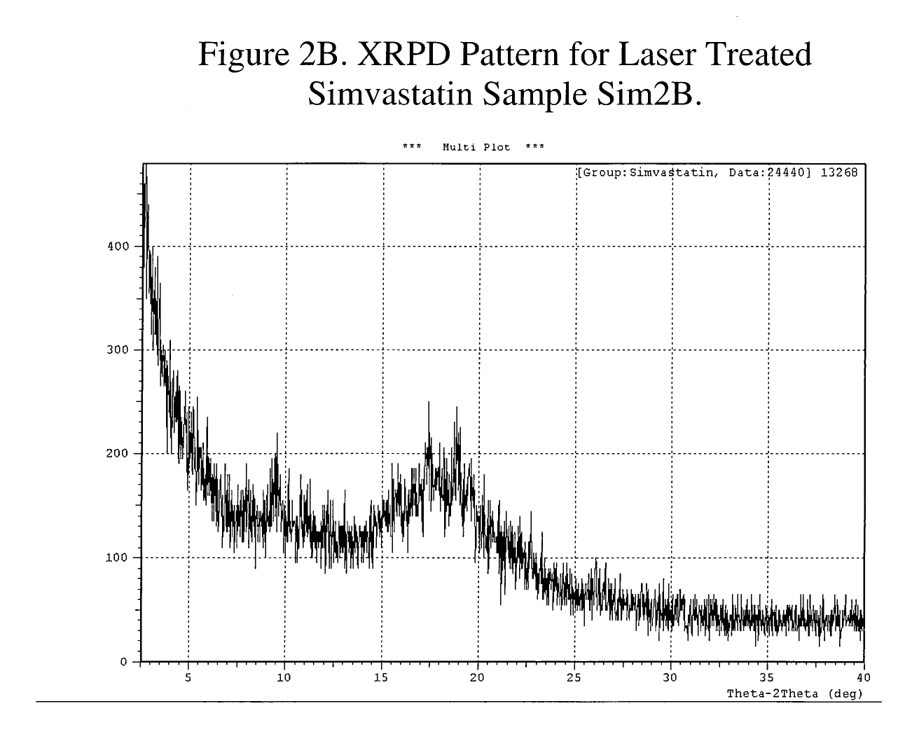

FIG. 2A shows the XRPD pattern for Sim 2A and FIG. 2B shows the XRPD pattern for Sim 2B. The XRPD pattern for Sim 2A shows relatively low intensity reflections and the XRPD pattern for Sim 2B displays very low intensity reflections. The low intensity reflections may be attributed to amorphous content, with the pattern of Sim 2B suggesting an even higher degree of amorphous content than Sim 2A.

Sim 2A and Sim 2B solidified in a glassy appearance with only slight crystalline development compared to the modestly developed crystal formations of Sim 1A and the highly developed crystal formations of Sim 1B. The degree of glassy appearance observed in Sim 2A and Sim 2B was consistent with the degree of amorphous content suggested by XRPD.

Amorphous materials generally have significantly higher free energy than crystalline materials of the same substance. Due to their greater energetic states, they tend to have higher solubilities and faster rates of dissolution than their less energetic crystalline counterparts. In many cases, the amorphous form of a pharmaceutical compound is chosen for clinical use because the lower solubility and bioavailability of the crystalline form limits the clinical value. Even in the case of simvastatin, it is possible that adding significant amorphous content to the composition may increase the rate of absorption and bioavailability, resulting in greater efficacy at a lower dose. If a lower dose proves sufficient for the desired clinical results, the likelihood of adverse effects may also decrease.

In contrast to the extreme conditions far from equilibrium that are often required to produce amorphous forms, laser acoustic resonance can achieve this formation at room temperature and pressure without drastic changes in pH. Avoidance of extreme conditions may reduce the degree of degradation of the compound that may occur under more aggressive conditions to improve product yield and perhaps result in a more stable amorphous form.

The application of laser acoustic resonance through modulated sparse constructive nodes may provide a means of reliably producing amorphous forms of compounds that are otherwise difficult to produce in an amorphous form. This may salvage compounds that would be likely to be clinically useful but do not otherwise achieve sufficient solubility to be effective. For other compounds, producing a stable amorphous content may increase bioavailability to the degree of increasing clinical efficacy, reducing dosage requirements, or decreasing the risk of adverse effects.

### EXAMPLE 3

### Increased Arginine-Derived Nitric Oxide Production through Laser Treatment

The use of laser modification of compounds enhances the ability to modify not only the compound itself, but also by-products created by the body's use of the modified compound.

For example, four Samples of L-arginine (Arg) of 20 grams each, 3 for laser treatment and 1 an untreated control, were measured. Arg #1 was treated with a Quantel Nd-YAG pulsed laser at 532 nm with an average pulse amplitude of 2-5 mJ/pulse over 5 nanoseconds, 12 pulses per second. The optics were adjusted to maximum cancellation and the sample was treated for 30 seconds. Arg #2 was treated with a 458 nm pumped argon gas laser with a primary power of 16.5 mW adjusted through the optics to a power level of 5.06 mW. Arg #3 was treated with a 670 nm diode laser of 4.85 mW primary power adjusted through the optical elements to a power level of 2.94 mW. Arg #4 was the untreated control sample.

Paracelsian in Ithaca, N.Y., an outside independent lab, performed the following bioassays. Each arginine sample was added to 12 wells of murine macrophages to achieve a concentration of 120 mcg/ml. This is the estimated serum concentration for a 70 Kg person after ingesting a 6-gram serving of arginine, a level observed in numerous clinical studies to be associated with a wide range of physiologic benefits. LPS at 1 ng/ml was added to each well and the cells were incubated for 24 hours. The nitrite concentration in each well's supernatant was determined 24 hours after initiation of treatment as a relative measure of nitric oxide production.

The results are listed in order of relative nitrite production, from greatest to least. The first column is the Arg #, the second the mean plus or minus the standard deviation of the optical density measurement at 540 nm, a measure of nitrite concentration, and the third column the relative production of nitrites expressed in micrograms per ml as determined from optical density. The final column shows the results of a Students 1-Tailed T-Test comparing the highest producing Arg #3 to the other samples.

| Sample ID | Mean O.D. ± S.D. | Nitrites | P Value |
|---|---|---|---|
| Arg #3 | .232 ± .010 | 12.0 | — |
| Arg #4 | .224 ± .006 | 11.4 | .0216 |
| Arg #2 | .219 ± .008 | 10.8 | .0016 |
| Arg #1 | .215 ± .007 | 10.6 | .0001 |

The laser modulation applied to Arg #3 resulted in this sample producing statistically significantly more nitric oxide byproducts than the control untreated Arg #4. The laser modulations applied to Arg #1 and Arg #2 resulted in their producing statistically significantly less nitric oxide byproducts than the control untreated Arg #4 and highly statistically significantly less than the laser activated Arg #3.

It is very important to note that the greatest effect of providing L-arginine on nitric oxide production in vitro and in vivo is probably within the first 30-60 minutes of delivery, so that a 24 hour equilibration survey may substantially underestimate the full magnitude of differential nitric oxide production of the laser treated versus control forms of L-arginine.

This example shows the ability to modify the production of the intended metabolic byproduct significantly upward or downward depending on the laser stimulus applied. The experiment was performed at an energy level too low to cause ionization or significant thermal degradation. It is most likely that changes in molecular shape that persist in effects even after material in the dry state goes into solution are moderating enzyme-substrate fit and reaction rates in the intended directions.

### EXAMPLE 4

### Homogenization, Elongation, and Dehydration of L-Arginine Hydrochloride through Laser Resonant Stimulation

In each of two 10×35mm polystyrene Petri dishes 135 mg of L-arginine Hydrochloride were measured. Each sample was dissolved in 0.50 grams of deionized water. The control sample was crystallized by slow evaporation at room temperature over 24 hours. The average room temperature was approximately 26 degrees Centigrade. The average ambient humidity was approximately 33%.

The treated sample was crystallized under the same conditions with the addition of pulsed modulated energy at 532 nm as described for Arg #1 in Example 2 above. The beam was passed through the center of the meniscus of solution in the container.

Crystals from the control and treated samples were selected for further study. Selected crystals all had dimensions of approximately 0.5 mm on a side or less as per the latest standards in the art. The crystal structures were solved using a SMART X-ray diffraction analytical device.

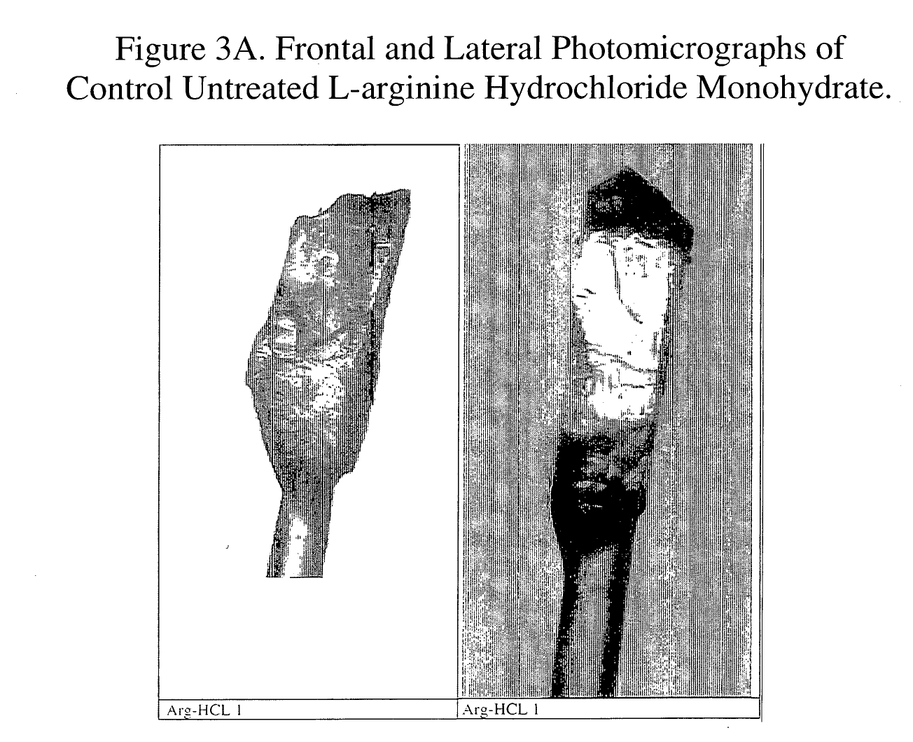

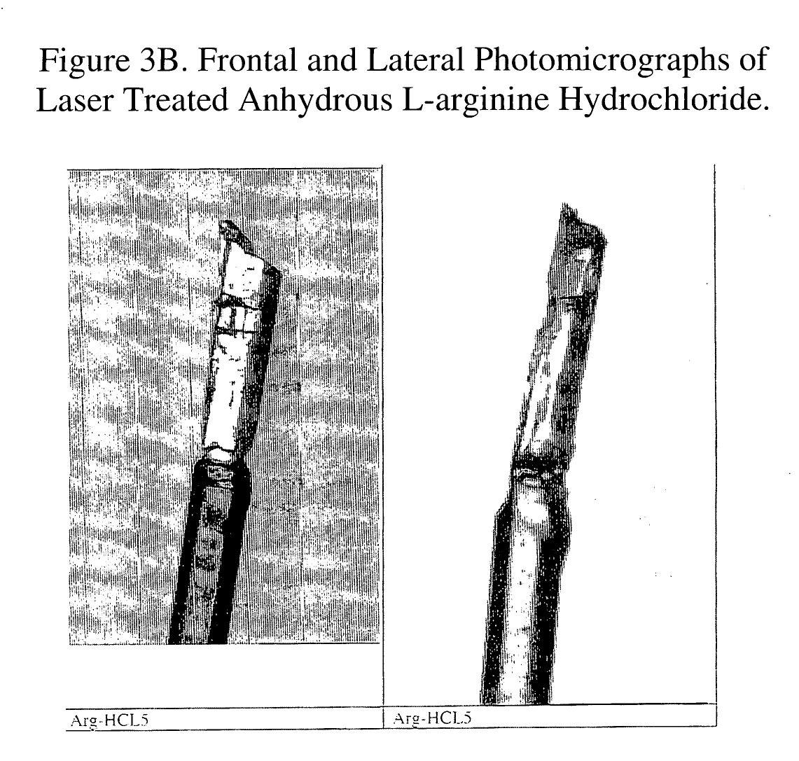

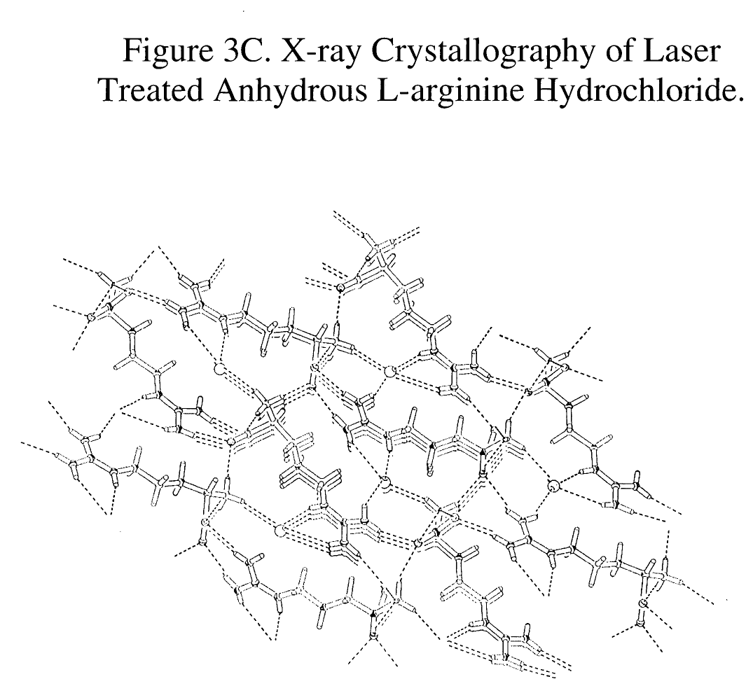

Highly significant differences were seen between the control and laser treated crystals. FIG. 3A shows the somewhat blocky and irregular habit of anterior and lateral views of the control sample; whereas FIG. 3B shows a more uniform cylindrical habit on comparable views for the laser treated L-arginine hydrochloride. The control L-arginine hydrochloride was found to have the typical unit cell characteristics of the monohydrated crystal reported in the literature. In contrast, the laser treated crystal was found to have a significantly different unit cell that was free of water in the crystal lattice, demonstrating the conversion of a monohydrate to an anhydrous crystal. This is particularly significant since the crystallization was done from water at room temperature. As shown in FIG. 3C, and consistent with the predicted effects of stimulating backbone resonances with sparse constructive nodes, there is a high level of homogenization of elongated L-arginine structures in the lattice.

The process described in this invention has the potential to be applied to a wide range of molecular forms to modify the relative intensities of hydrophilic and hydrophobic interactions. Material in the dry state can be pre-treated to upregulate or downregulate specific reaction processes in the directions intended. Crystals grown from solution using this process may have novel and desirable properties. This process may also be applied in solution to modify reaction rates and product ratios. Greater depth of penetration through media of sparse constructive nodes of laser EM waves can extend this process to a broad range of industrial, in vitro, and in vivo applications.

### EXAMPLE 5

### Reduced Production of Inflammatory Cytokines through Laser Treatment of a Complete Spectrum Blend of Amino Acids

A mixture of amino acids was prepared as follows: Dry powders of the following free form amino acids were measured and mixed in the following proportions: L-cysteine 3.4 grams, L-taurine 6.8 grams, L-threonine 27.0 grams, glycine 368.4 grams, L-glutamic acid base 67.6 grams, L-glutamine 67.6 grams, L-lysine monohydrochloride 67.6 grams, L-arginine 60.8 grams, L-aspartic acid 13.6 grams, L-omithine monohydrochloride 12.2 grams, L-histidine 13.6 grams, L-leucine 60.8 grams, L-valine 33.8 grams, L-methionine 33.8 grams, DL-phenylalanine 129.0 grams, L-isoleucine 40.6 grams, L-alanine 16.8 grams, L-proline 13.6 grams, L-serine 33.8 grams, and L-citrulline 10.2 grams.

Twenty (20) grams each of this mixture were used for control and laser treated samples. Sample 1 was the control, Sample 2 was treated with a 670 nm diode laser of 4.85 mW primary power adjusted through the optical elements to a power level of 2.94 mW, and Sample 3 was treated with a 458 nm pumped argon gas laser with a primary power of 16.5 mW adjusted through the optics to a power level of 5.06 mW. Durations of laser treatments for Samples 2 and 3 were 30 seconds each.

An independent outside lab, Paracelsian in Ithaca, N.Y., performed the following bioassays. A standardized Echinacea sample alone or with 20 mg/ml of Samples 1, 2, or 3 were incubated in the tissue culture media of triplicate wells of murine macrophages for 24 hours after Echinacea stimulation and then assayed for tumor necrosis factor-alpha (TNF-alpha) production in triplicate ELISA wells. Positive controls with lipopolysaccharide (LPS) at 1 ng/ml and negative controls were also assayed in the same manner.

Those skilled in the art will appreciate that the use of murine macrophages simulates the body's immune response. Adding the herb Echinacea provides a similar response to that of an immune system that is being irritated. A TNF-alpha reading is a good marker for the extent of inflammation. Thus a substance that causes a significant increase in TNF-alpha in the macrophages can be expected to create substantial inflammation in a human body—especially a body suffering from an autoimmune disease such as inflammatory bowel disease, as well as other physiological problems such as systemic lupus erythematosus, rheumatoid arthritis and food allergies.

The results were as follows:

| Sample ID | TNF-alpha ± S.D. |
|---|---|
| Negative Control | 215 ± 13.7 |
| Echinacea Positive Control | 683 ± 27.1 |
| LPS 1 ng/ml Control | 2863 ± 185.7 |
| Echinacea + Sample 1 | 1568 ± 45.8 |
| Echinacea + Sample 2 | 850 ± 57.6 |
| Echinacea + Sample 3 | 761 ± 100.3 |

Using a Students 2-Tailed T-Test, the Echinacea positive control was compared to the results of Echinacea plus Samples 1, 2, or 3. The addition of Sample 1 resulted in a highly significant increase in TNF-alpha at p<0.0001. The relative increase in TNF-alpha production was not as great after the addition of Sample 2, but was still statistically significant at p<0.03. The addition of Sample 3 did not significantly increase TNF-alpha production, with p=0.31. Thus the laser treatment of Sample 3 reduced the robust increased production of TNF-alpha observed with control Sample 1 back to the baseline level of Echinacea alone.

In other words, the negative control is indicative of the immune system of a normal person. Adding the Echinacea heightened the immune response. The addition of lipopolysaccharide (LPS) simulates a maximum immune stimulus, as a point of reference.

The addition of Sample 1, the unmodified amino acids, showed a marked increase in TNF-alpha production. Thus, a person with an autoimmune disease, or other inflammatory processes, would expect to have substantial inflammation as a result of ingesting the amino acids.

In contrast to Sample 1, Sample 2 and Sample 3 were modified as set forth above. Not only did the Samples not create a strong likelihood of inflammation, as did Sample 1, the increase in TNF-alpha was very minor. In fact, Sample 3 showed virtually no increase in inflammation of the Echinacea positive control.

Those familiar with nutrition will appreciate that many people have difficulty tolerating certain nutrients that are required for good health. The amino acids discussed above are a prime example. By subjecting a wide variety of amino acids to laser treatment, the bioavailability of the amino acids can be greatly increased. Obviously, if a person with an autoimmune disorder or other inflammatory condition does not react negatively to the amino acids, considerably more can be incorporated into the person's diet without risk of unwanted side effects.

Those skilled in the art will appreciate that inflammation is not always a bad thing. There are many times when a heightened immunological response may be desired. For example, increased inflammation/immunologic activity may be used to fight tumors, or other undesirable conditions. By modifying the laser treatment of chemicals, the chemicals can be altered so that they increase immunologic response, rather than minimize the same, as already demonstrated through increased nitric oxide production of macrophages from laser treated versus untreated control L-arginine.

### EXAMPLE 6

### Improved Brain Coherence Using Laser Homogenized Versus Untreated Amino Acids

An electroencephalogram (EEG) is a diagnostic study that places recording electrodes over the brain to measure the pattern of electrical activity in the brain. A quantitative EEG, or brain map, is a detailed study that measures the power of brainwaves in the frequency bands delta, theta, alpha, and beta, with power expressed in microvolts. In addition, a brain map also measures coherence, which refers to whether the phases of the brainwaves from one region to another are in a relationship consistent with healthy versus disordered brain function.

The standard conditions for a quantitative EEG are in the morning after a good night's sleep, with avoidance of caffeine and other stimulants. A cap with conductive electrodes is placed over the scalp such that the electrodes localize over specific regional brain areas. Measurements are taken with the eyes closed and the subject resting supine for a period of 20-30 minutes. If baseline and post intervention measures are done, the same protocol is followed with the cap left in place to insure reliability of localization from measurement to measurement. Resting with closed eyes tends to cause significant augmentation of the alpha wave band at 8-12 cycles per second, making this wave band of particular significance for study interpretation. The quantitative EEG equipment used for the following studies measured power output and coherence data over 19 different locations over the brain.

The study test formula consisted of a blend of amino acids intended to increase mental energy, concentration, and alertness. The two most important amino acids for increased brain energy and alertness are L-phenylalanine and L-tyrosine, as these are the precursors to the catecholamine neurotransmitters dopamine, norepinephrine, and epinephrine. In the standard chemical pathway, L-phenylalanine is hydroxylated to L-tyrosine, which is then itself hydroxylated to form L-dopa. The enzyme L-dopa decarboxylase then converts L-dopa to dopamine; differential hydroxylation of dopamine can then yield either norepinephrine or epinephrine, the other major catecholamine neurotransmitters that can have profoundly stimulant effects in the central nervous system and systemically.

The study formula was composed of the following ingredients as percentages by weight: L-tyrosine 6.6%, L-phenylalanine 3.3%, DL-phenylalanine 2.2%, glycine 4.4%, L-arginine 7.7%, L-omithine 7.7%, L-lysine 3.3%, L-taurine 6.6%, L-glutathione 9.9%, L-glutamic acid 5.5%, L-glutamine 4.4%, L-methionine 4.4%, L-cystine 7.7%, L-cysteine 3.3%, L-alanine 5.5%, L-threonine 2.2%, L-valine 6.6%, L-isoleucine 4.4%, L-leucine 1.1% L-histidine 2.2%, and L-aspartic acid 1.1%. Into clear gelatin capsules, 750 mg of the study formula were placed per capsule. The control untreated capsules received no further modification. The laser treated capsules were irradiated with pumped argon laser light at 458 nm, as in Example 1 for Arginine #3. The treated capsules were slowly rotated through the beam for one minute per capsule.

The subjects were two young adult white females with no known medical problems and no history of brain injuries or neurologic illness. They were chosen as subjects to represent the brain physiologic responses of young healthy persons. As anticipated the baseline quantitative EEGs showed a preponderance of alpha waves as expected for the resting state with eyes closed. After taking the baseline readings the subjects were each given 2 capsules of the untreated study formula, 1.5 grams total per subject. After 30 minutes to permit absorption and assimilation of the formula, quantitative EEG measurements were repeated. Following the measurements of the untreated study formula, the subjects then ingested two capsules of the laser treated formula, 1.5 grams total per subject. After 30 minutes to permit absorption and assimilation of the treated formula, quantitative EEG measurements were again repeated.

The following table shows the mean and standard deviation (SD) values for the power output in the alpha band for baseline, post untreated amino acids, and post treated amino acids.

| | Mean | Standard |
|---|---|---|
| | (Microvolts) | Deviation |
| Baseline | 8.339 | 4.876 |
| Untreated Amino Acids | 11.2842 | 6.7014 |
| Treated Amino Acids | 11.9842 | 8.2596 |

A General Linear Model repeated measures analysis of variance was used to analyze the effect of a laser energized amino acid formula on enhancing brain power, comparing baseline, ingestion of the untreated formula, and ingestion of the laser treated formula. Multivariate testing indicated a significant increase in brain power over baseline with Wilks' Lambda=0.219, F (2,36)=64.128, p≦0.0001. Paired two- tailed t-test analysis indicated significant increases in brain power were found over baseline after ingesting both the untreated and laser treated amino acids, both comparisons statistically significant at p<0.0001. Further, the laser treated amino acid formula significantly increased brain power over the untreated formula, with t (37)=−2.349, p=0.024.

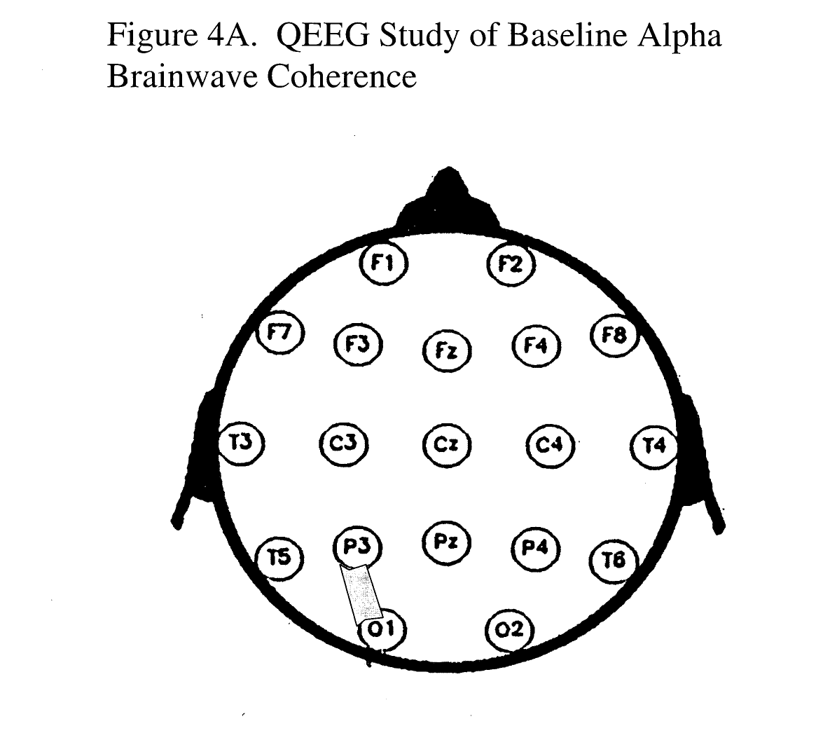

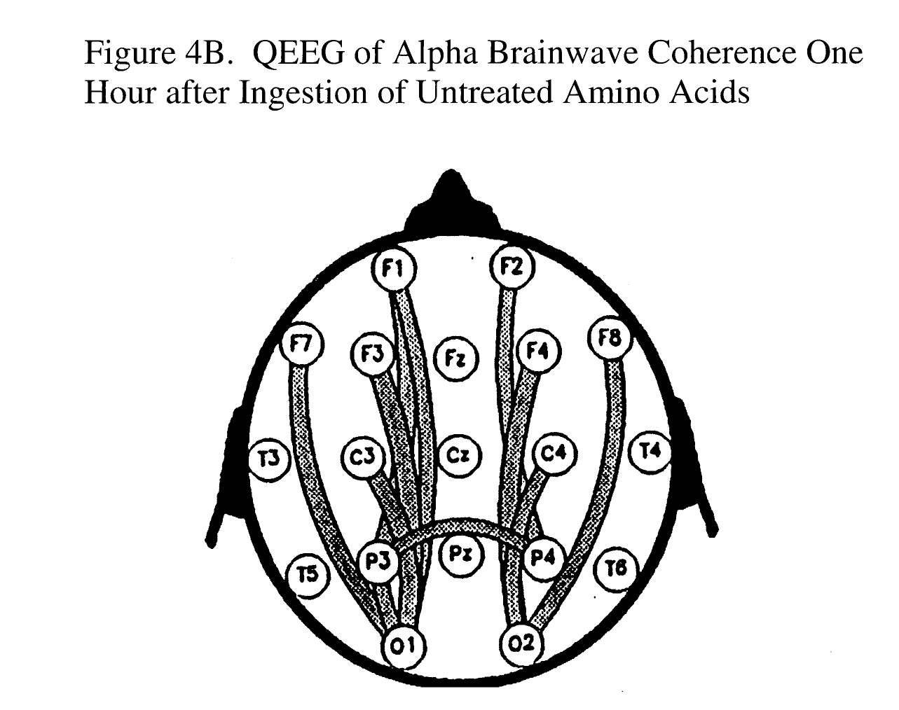

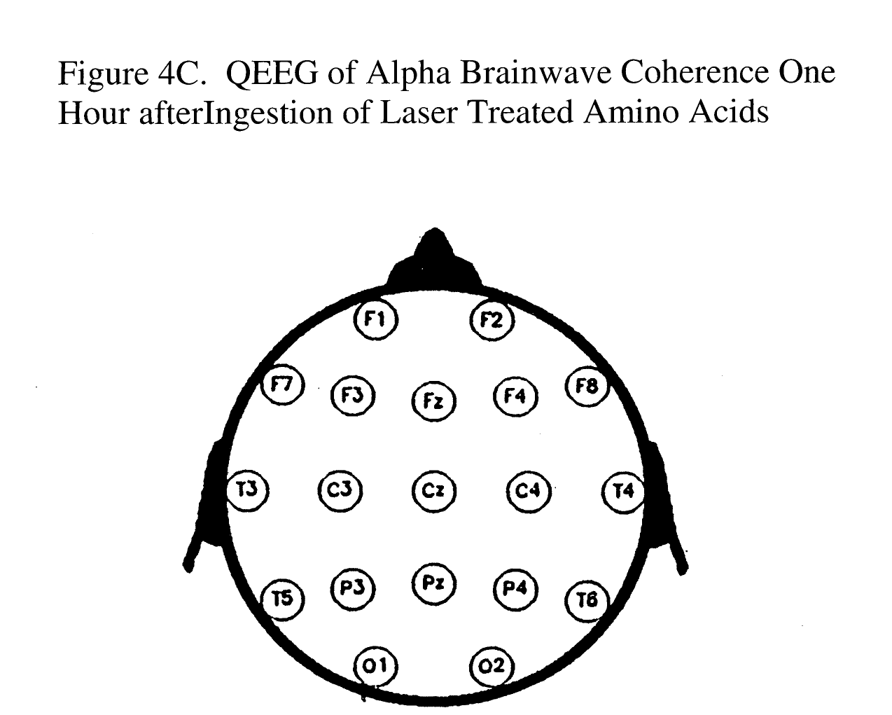

In addition, use of the laser treated formula also showed a significantly better coherence result than the use of the untreated amino acid formula. One of the two subjects showed the adverse effect of a significant degradation of her brainwave coherence in alpha after the ingestion of the untreated amino acids that improved to better than baseline after ingesting the laser treated amino acids. FIG. 4A shows the baseline coherence study for this subject that demonstrates a single brainwave coherence abnormality in the left posterior region of the brain. Following the ingestion of the untreated amino acid formula, FIG. 4B shows the development of extensive coherence abnormalities. From a single defect at baseline, 11 regions of abnormality have developed that show intense front to back coherence defects bilaterally, with a region of interhemispheric coherence defect as well. Following the ingestion of the laser treated amino acids, FIG. 4C shows the complete resolution of all coherence defects. Use of the laser treated amino acids not only showed the ability to increase the power output of the brain significantly over the untreated amino acids, but also showed the ability to reverse the adverse effect of abnormal brainwave coherence that occurred with the use of the untreated amino acids. It is possible that inhomogeneities in shape and backbone twist, particularly of the precursors to the phenolic neurotransmitters, may predispose to inconsistent receptor effects and suboptimal neurophysiologic responses.

In the commercial production of L-tyrosine, the heating and dehydration processes to which the molecules are subjected, particularly pulling water molecules from the structure, may result in twisting the phenol ring on the backbone chain or other distortions of the molecular shape. For the untreated L-tyrosine, those shapes that failed to provide optimum receptor fit for its catecholamine neurotransmitter metabolites may be a factor in the development of coherence abnormalities. The homogenization of the configuration of the laser treated L-phenylalanine and L-tyrosine could be a key factor promoting the restoring of normal brain coherence that may occur through improved receptor fit of neurotransmitters, while also sustaining increased brain energy.

Likewise, L-dopa is subjected to thermal and dehydration stresses during its commercial manufacture. These stresses may also result in molecular distortions of the phenolic ring alignment on the backbone. Widely used as a pharmaceutical agent to treat Parkinson's disease, L-dopa provides the substrate to increase dopamine levels deficient in specific brain regions (especially the substantia nigra and other striatal nuclei) in that condition. L-dopa is usually given with carbidopa, an inhibitor of dopa decarboxylase outside of the brain so that higher concentrations of L-dopa cross the blood-brain barrier.

Although L-dopa may help to relieve the movement disorders of Parkinson's disease, its use is frequently complicated by side effects such as nausea and agitation. Because of diminishing efficacy, escalating doses are often required, which also tends to further increase side effects, which may become dose limiting. The use of laser resonance to homogenize L-dopa may yield a shape that more consistently promotes the intended clinical effects, while reducing the side effect profile. It may be possible that a given dose of laser treated L-dopa will provide equivalent or greater clinical benefits, may reduce the tendency of adverse effects, and delay the requirement of dosage escalation. The initial protocol to use for the laser treatment of L-dopa would follow the practice used for treating the amino acid formula as above, scaled up for higher volume powder delivery as suited to commercial production levels.

### EXAMPLE 7

### Increased Quality of Crystal Formation with Flattened and Stretched Carbon-Nitrogen, Carbon-Hydrogen, and Carbon-Oxygen Bonds in Laser Treated Versus Untreated Betaine Hydrochloride

In accordance with the present invention sparse constructive node laser irradiation has been used to resonate betaine hydrochloride molecules to a homogeneous flattened and stretched shape. The homogenization effect is observed at the level of much improved crystal formation of the laser treated betaine hydrochloride versus the untreated control. X-ray crystallography of the laser treated betaine hydrochloride shows the predicted flattening and stretching of the bonds in the treated molecules compared to the control untreated molecules.

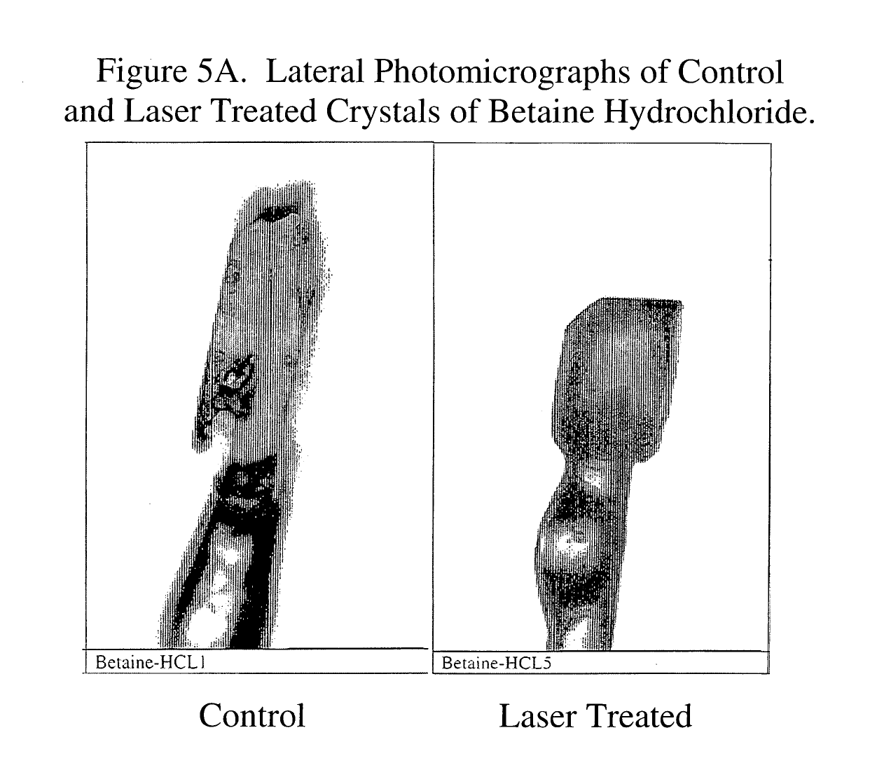

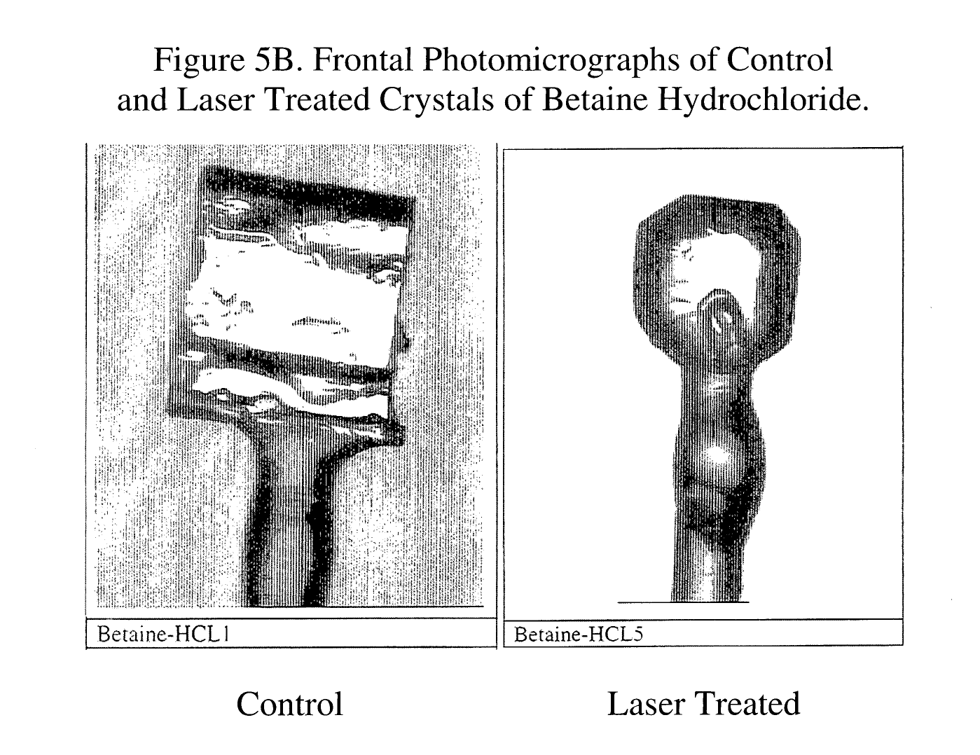

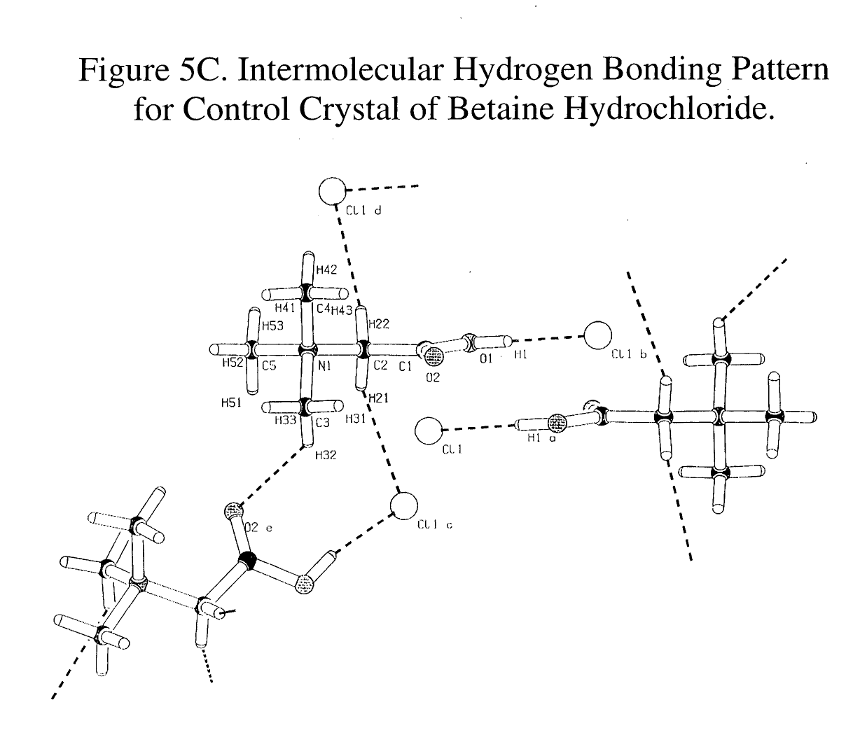

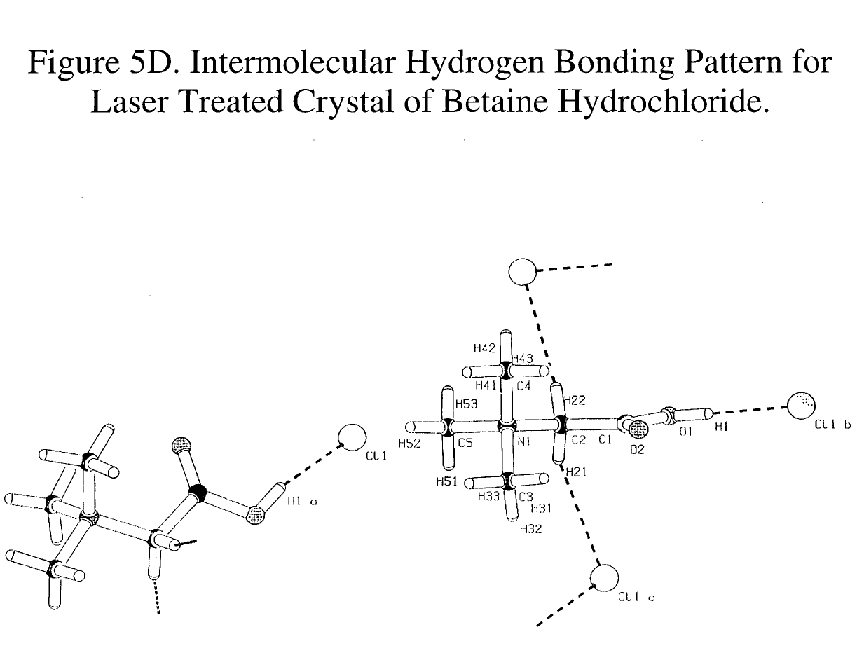

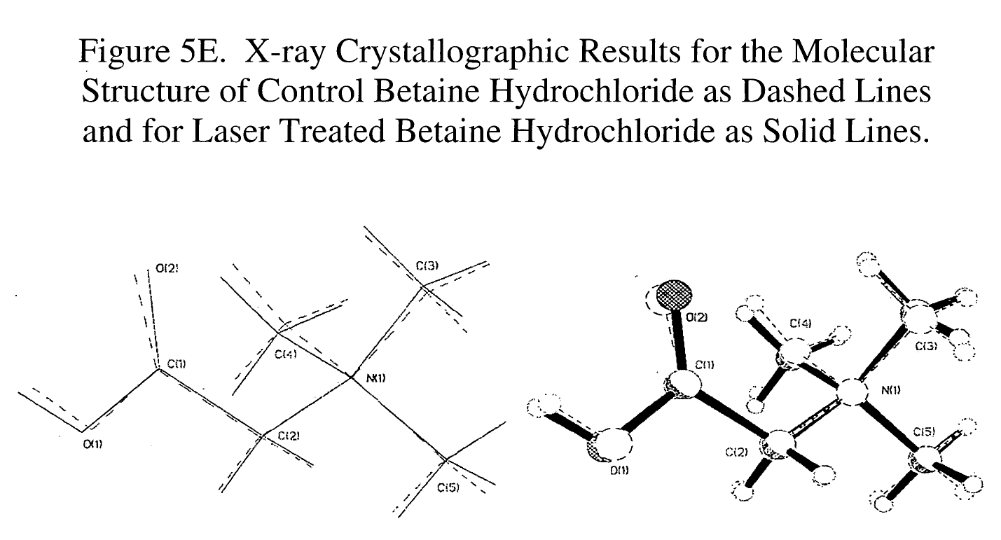

The control and treated betaine hydrochloride samples shown in FIGS. 5A, 5B, 5C, 5D, and 5E were prepared by dissolving 0.6 grams of betaine hydrochloride in 3.0 grams of deionized water and placing the solutions thus prepared in 10×35 mm Petri dishes. Crystallization was done in open containers by slow evaporation at room temperature, a procedure often used in the art of crystallography. Ambient humidity was maintained at or below 30 percent with laboratory dehumidifiers. The treated betaine hydrochloride was irradiated with a 670 nm continuous wave diode laser modulated at 10 MHz with a primary beam power of 2.7 milliwatts that was phase conjugated to 1.35 milliwatts. The 5 mm diameter beam was passed through the middle of fluid meniscus of the treated solution during the entire crystallization process. The control untreated betaine hydrochloride was prepared under the same conditions, except that it was not irradiated with the sparse constructive node generating laser system.

The quality of crystal formation in control versus laser treated betaine hydrochloride is shown in FIGS. 5A and 5B. The crystallographic term for the overall geometric shape of the crystal that has formed is the crystal habit. In FIGS. 5A and 5B the control crystals on the left have a markedly different habit from the treated crystals on the right.

The magnified lateral view photographs of 5A show a significant difference between the control and laser treated betaine hydrochloride. The control crystal has numerous inclusion defects, surface irregularities and much shallower depth. In contrast, the treated crystal shows a high level of uniformity, free of defects, with a smooth surface, and a greater front to back depth. The frontal views of 5B show a wavy, irregular surface of the control crystal with a coarse outline of the edges. In comparison, the laser treated crystal on the right shows a much smoother surface with smoother contours of the edges.

These figures demonstrate the process of homogenization. Betaine hydrochloride tends to have a backbone twist, resulting in a range of shapes in solution. Even with a slow evaporation over several hours, the differential of shapes prohibited an orderly arrangement in the crystal lattice, leaving gaps and irregularities in the crystal. As slightly different shapes were added to the growth zone of the crystal, the growth planes were distorted resulting in crystal irregularities.

In contrast, the betaine hydrochloride grown under the influence of laser homogenization achieved such self-similarity that a highly organized crystal free of gross defects was formed. The slight heating of the medium that may have resulted from low power laser application would, if anything, have tended to cause less organization, which was overridden by the sparse constructive node effects.

It is important to note that the production of the control crystal by slow evaporation is a very gentle process compared to the usual modes of industrial drying of bulk quantities of product. Typically much higher temperatures are used, up to the threshold of thermal degradation of the compound.

Such aggressive conditions will substantially increase the tendency for more widespread and extreme distortion of molecular structure through random thermal motion and greater intensity of dehydration. Sparse constructive node laser irradiation can be applied to dried powders (as in Examples 1, 3 and 4) or during the dehydration process to homogenize molecular shapes and thereby improve bioavailability.

X-ray crystallography was performed using a SMART device made by Siemens. FIGS. 5C and 5D show the intermolecular hydrogen bonding of control versus laser treated betaine hydrochloride, respectively. FIG. 5C shows 4 intermolecular hydrogen bonds per molecule of untreated betaine hydrochloride. In comparison, FIG. 5D shows only 3 intermolecular hydrogen bonds for each molecule of treated betaine hydrochloride. Although this is a soft feature of the crystallography, reducing the number of hydrogen bonds can increase solubility; faster dissolving of substrate into solution could promote more rapid absorption of the molecule.

FIG. 5E shows the crystal solution for control and laser treated betaine hydrochloride through x-ray crystallography. The crystallographic solution refers to the process of using the x-ray diffraction pattern to determine the precise localization of all of the atoms in the molecule being analyzed.

In the two diagrams, the dashed lines show the structure of control untreated betaine hydrochloride, whereas the solid lines show the structure of the laser treated betaine hydrochloride. The upper diagram shows backbone model representations and the lower diagram shows ball and stick model representations. In both diagrams, the treated betaine hydrochloride shows the predicted effects of flattening and stretching of the molecule. In particular, there is flattening and stretching of carbon-nitrogen bonds (of the methyl groups), carbon-oxygen bonds, and to a lesser degree carbon-hydrogen bonds.

Homogenization and molecular flattening and stretching can increase the efficiency of enzyme moderated reactions through at least three basic mechanisms, thereby enhancing bioavailability. Increasing the homogenization of the substrate is analogous to increasing the concentration of the substrate for the isoform of the enzyme preferred for that substrate. In any enzyme moderated reaction increased substrate concentration will proportionately increase reaction rates and product generation.

Secondly the flattest shape will tend to be the lowest energy state that is homogeneous. In this configuration bond strength is lowest, while field strength is highest. This is a very reactive state as the substrates behave as whole molecules.

In addition, the high self-similarity from molecule to molecule facilitates enzyme binding, because enzymes will bond considerably faster to a molecule identical to that just released than to one even slightly dimensionally different. This means that the rate at which the reactant can be supplied is directly proportional (times a constant) to the self-similarity of the molecules in the reactant. Thus cells will make more product the more similar the molecules of the reactant are to each other with respect to dimensional shape and water distribution. Molecules exposed to sparse node irradiation will in general be highly similar in terms of water distribution and location, will tend to have the flattest low energy shape possible with a high electrical and magnetic moment, and will be extremely self-similar in all dimensions.

The betaine hydrochloride crystals made from exposed and unexposed betaine hydrochloride showed this effect to a marked degree because small individual differences add up to larger macroscopic differences visible in a grown crystal. Self-similarity also reduces the need for a cell to manufacture a wider range of enzymes to moderate a given reaction than is the case if the cell is presented with a highly inhomogeneously crystallized reactant with widely varying shapes. Increasing the similarity of bond energy and dimension molecule to molecule will generally tend to favor the production of any product in an enzyme moderated reaction, and thereby increase bioavailability.

The process of cells making a product can be viewed as a manufacturing process where the cells take in raw materials at one end with the aim of producing a specific product at the other. The concept of a nutritional supplement at the fundamental level is to make available raw materials for a given product that would otherwise require prior reactions to extract from available foodstuffs. Thus the principle of a nutritional supplement is to reduce the reaction complexity of a given product and hence the energy and time required to produce it.

Increasing the homogeneity of the nutritional supplement is simply an enhancement of that same principle further reducing the complexity of the reaction and increasing the speed and efficiency of producing the desired product, thus enhancing bioavailability over unhomogenized nutrients. Likewise a pharmaceutical agent intended to increase a desired product in the body through enzyme moderated reactions, such as producing dopamine from L-dopa, may also show enhanced bioavailability and potentially fewer side effects if laser homogenized rather than if untreated pharmaceutical agents are used.

In addition, pharmaceutical agents that are designed to increase receptor activity (such as the beta-blocker propranolol) may show similar shape moderated effects of bioactivity. It is well known that receptor—ligand fit is highly shape dependent. Homogenization to a highly self-similar flattened shape with a high electric and magnetic field moment may similarly function as though increasing ligand concentration for the desired receptor-ligand effect. This may permit both lower dosing for similar clinical benefits as well as reduced adverse effects at similar dosing levels.

### EXAMPLE 8

### Clinical Effects of Laser Treated Betaine to Reduce Homocysteine and Improve Clinical Symptoms

A randomized prospective placebo controlled double blind study was performed to determine the effects of laser treated betaine plus metabolic cofactors on methylation metabolism and clinical symptoms. Methylation metabolism refers to the transfer of methyl groups, the simple organic chemical group consisting of a carbon atom bonded to three hydrogen atoms (CH₃).

Also known as single carbon transfers, methyl group transfers are among the most fundamental and important chemical transfers in cell biology. Methyl group transfers are involved in the manufacture of DNA, the repair and maintenance of cell membranes, synthesis and balance of neurotransmitters in the central nervous system, and numerous other processes that modify proteins, lipids, and sugars into their biologically useful configurations.

Methylation metabolism is also intimately involved in DNA regulation and biological timing mechanisms. Given the widespread importance of methyl group transfer metabolism, therapeutic agents that enhance methyl metabolism would be expected to have significant potential for improving overall metabolic balance and related clinical conditions.

A key indication of the integrity of methyl metabolism in the body is the homocysteine level. Elevation of serum homocysteine indicates impairment of one or more of the main methyl metabolism pathways. Elevated homocysteine is also clinically relevant. Published epidemiologic data indicates an exponential rise in the relative risk of cardiovascular disease for homocysteine levels above 6.3, as shown in the following chart:

| | | | | | |
|---|---|---|---|---|---|
| Homocysteine Level | <6.3 | 6.3 | 10 | 15 | 20 |
| Relative Cardiac Risk | <1 | 1 | 2 | 4 | 9 |

In addition, elevated homocysteine has been associated with an increased risk of stroke, Alzheimer's disease, pre-eclampsia, neural tube birth defects, fetal loss, human hostility, and the development of malignancies. In homocystinuria, a metabolic disorder in which homocysteine can rise into the hundreds, accelerated aging, neurologic disease, and atherosclerosis can be highly aggressive even at early ages.

Homocysteine is produced in the body as a byproduct of metabolism of the amino acid methionine. There are three main pathways the body uses to clear homocysteine that when effective can prevent its rise to hazardous levels.

The first pathway is the transsulfuration pathway that uses vitamin B6 (pyridoxine) and zinc to detoxify homocysteine to the amino acid cysteine. Methionine and cysteine are the main sulfur containing amino acids, and methionine can be converted to cysteine via homocysteine if their pathways are intact. Some persons are unable to phosphorylate pyridoxine to its activated state; in these persons, pyridoxal-5′-phosphate must be given to overcome the metabolic block.

The second homocysteine detoxification pathway uses vitamin B12 and folic acid to remethylate homocysteine back to methionine. Deficiencies of B12 and folic acid are well known to result in neurologic, psychiatric, and hematologic defects. Disturbed methyl group transfer metabolism impairs the synthesis of DNA, neurotransmitters, and myelin that can result in anemia, dementia, psychiatric disease, and peripheral neuropathies. Deficiencies of folic acid in particular have been associated with an increased risk of colon and cervical cancer, as well as birth defects of the central nervous system. Genetic defects of this pathway are common in certain populations, e.g., 38% of French Canadians are heterozygous for defective activity of the enzyme methyltetrahydrofolate reductase. Aggressive support of the complete methyl metabolic pathways may significantly reduce the health hazards of such inborn metabolic defects.

The third pathway for clearing homocysteine, and perhaps the most powerful clinically, uses betaine as a methyl group donor. Through betaine-homocysteine methyltransferase, an enzyme found in the liver and kidneys, a methyl group from betaine is transferred to homocysteine to convert it into the essential amino acid methionine. Betaine itself is a derivative of the amino acid glycine that has had its three amino hydrogen atoms replaced with three methyl groups; thus betaine is a methyl group rich methyl group donor also known as N,N,N-trimethylglycine, or simply as TMG.

A double blind clinical study conducted by Morrsion et al. in 1953 looked at the effects of administering methyl group transfer factors on subjects that had just survived a first myocardial infarction. Treated subjects received high-dose betaine of 9 grams daily plus vitamin B12, a liver extract, and a creatine precursor. After one year, subjects given the placebo had 25% mortality versus no mortality in the treatment group, which was a highly significant reduction of mortality in the treatment group.

Persons with homocystinuria, the most extreme scenario of disturbed methyl metabolism, may also show a reduction of homocysteine level with vitamins B6, B12, and folic acid, but often do not have a significant improvement in clinical condition. In contrast, adding high dose betaine (typically 6-9 grams daily) has been associated with reversal of graying hair, improved cardiovascular status and even reversal of neurological defects. Women with homocystinuria have been able to conceive and have normal gestation and term deliveries when betaine has been added to their regimen.

Ingestion of betaine has also been associated with reduced body fat, increased muscle mass, and enhanced athletic performance. Betaine also plays a role in intracellular osmotic regulation, especially in the kidney.

The generation of methionine, particularly in the liver, sets the stage for one of the most important processes of methyl metabolism. A molecule of methionine combines with the energy molecule ATP (adenosine triphosphate) to form the molecule S-adenosyl-methionine (SAMe), through the action of the enzyme SAMe synthetase. The SAMe thus formed is the predominant methyl group donor in cellular metabolism, involved in several dozen methyl group transfer reactions.

In particular, all of the DNA methyltransferases, the enzymes that regulate DNA transcription, aging, and repair through DNA methylation, exclusively use SAMe as the DNA methyl group donor. In addition, SAMe donates methyl groups to proteins, lipids, and carbohydrates to modify them into their biologically active configuration. Membrane lipids in particular require methylation for optimum fluidity and receptor function.

From the neurologic standpoint, SAMe provides methyl groups for neurotransmitter synthesis and balance, particularly the synthesis of serotonin, as well as for production of the insulating myelin sheaths of nerves.

Double blind clinical studies using ingested SAMe have documented several therapeutic benefits. At doses of 1600 mg per day, anti-depressant effects comparable to tricyclic anti-depressant drugs have been seen. In contrast to tricyclic pharmaceutical agents, the anti-depressant effects of SAMe were seen within one week as opposed to the usual 4-6 weeks required to achieve clinical benefits with tricyclic medications. In addition, the use of SAMe was essentially free of side effects, as opposed to the frequent anti-cholinergic and cardiovascular adverse effects observed with tricyclic pharmaceutical agents.

Other reported clinical benefits of SAMe include reduced pain and increased function in osteoarthritis, reduced symptoms of fibromyalgia, and improved cardiovascular health. SAMe use has also been reported to protect the liver from toxins and promote liver repair, even of cirrhosis. The latter effects are likely related to SAMe enhancing methylation in the liver, an important pathway of detoxification.

Once SAMe donates its methyl group, it then becomes S-adenosyl-homocysteine (SAH). Upon release of the adenosyl group, homocysteine is the resultant byproduct. FIG. 6A shows the general outline of the methyl group transfer pathways. Although administration of SAMe has been associated with clinical benefits, it has the potential drawback of increasing the homocysteine load.

A more ideal method of optimizing methyl metabolism would be to increase endogenous SAMe production while reducing homocysteine levels, as long as SAMe can be sufficiently boosted. Betaine administration is a strong candidate for raising SAMe while reducing homocysteine, as animal studies have shown that giving betaine may raise liver SAMe levels up to fourfold. Consistent with the betaine results on raising liver SAMe levels, giving betaine has also been shown to protect the liver from the adverse effect of toxins, in particular protecting the liver from alcohol induced toxicity.

Supportive of this role for betaine, a case study of a young woman with severe homocystinuria and major neurologic defects showed marked resolution of neurologic deficits when betaine was added, but not with vitamin administration alone; in addition her cerebrospinal fluid SAMe levels rose from nearly undetectable to normal levels, in conjunction with clinical improvement upon addition of betaine to her regimen.

As a pilot test, a female subject with osteoarthritis had blood SAMe levels measured while taking SAMe and then while taking a betaine formulation. The subject was taking 800 mg of SAMe daily, which provided a moderate degree of relief from knee pain. On this level of SAMe ingestion for three months, her blood SAMe level was 4.9 (the normal range for this lab is 4.2-8.2). At this time, SAMe was discontinued and she started a methylation formula with one gram of laser treated betaine plus laser treated metabolic cofactors. The betaine and metabolic co-factors were in the same ratios as in the double blind clinical study formula to be described below.

After one month, her blood SAMe level had risen to 6.2 and her right knee pain had nearly fully resolved. Thus giving the precursor of SAMe rather than SAMe itself resulted in a significantly higher blood level of SAMe and a greater clinical response. In particular, the dual effect of raising SAMe while reducing homocysteine would be expected to preserve and improve the condition of DNA methylation, as SAMe is the exclusive methyl group donor for DNA methylation.

Elevation of homocysteine has been found to be the most reliable marker of impaired DNA methylation, other than the direct measurement of DNA methylation status. Elevated homocysteine has also been associated with accelerated shortening of the telomeres in vascular endothelial cells. Telomeres are the ends of chromosomes that tend to shorten with each cellular division. When telomeres shorten excessively, the cells tend to lose the ability to replicate. Homocysteine elevation is thus associated with two fundamental DNA aging mechanisms; reducing homocysteine would therefore be expected to have significant effects supporting life extension.

The pattern of DNA methylation at birth is vitally important to the integrity of function of each type of cell. Methyl groups are placed on specific cytosine residues to differentiate the DNA expression of each cell type, through blocking the transcription of genes not appropriate to be produced in that cell line. The methyl groups on specific cytosine residues thus serve as regulatory blocks to prevent expression of genes inappropriate for that cell type. This mechanism, for example, prevents brain cells from making muscle proteins and muscle cells from making proteins that would be the exclusive province of brain cells. Every cell line therefore has a particular pattern of which residues in the genome undergo cytosine methylation.

This methylation pattern thus serves as a type of fingerprint that differentiates one cell line from another through blockade of transcribing gene products not suitable to that cell line. Cytosine methylation is a central regulatory process that determines which of the approximately 100,000 genes in the human genome will be expressed in a particular cell line.

The gradual loss of methyl groups from DNA is one of the most important timing mechanisms for aging and DNA degradation in the cell. At birth, depending on the type of cell, the cytosine methylation level ranges from 2-6% of the cytosine residues. The highest level of DNA methylation in humans and other mammals is typically seen in the thymus gland, with a cytosine methylation level of 6%. As methyl groups are gradually lost from DNA, integrity of transcription and DNA regulation is reduced. The DNA may begin to transcribe inappropriate genes for that particular cell line. Oncogenes may lose the suppressive effect of methylation and be at risk for activation, a change that may increase the likelihood of tumor formation. The cell chemistry associated with impaired methylation then increases the risk of DNA strand breaks and mutations.

At least in part due to the DNA changes associated with demethylation, a 20% loss of methyl groups from birth is associated with a significant increase in the risk of certain malignancies, particularly colon and cervical. Looking at the single variable of folic acid, persons with high versus low folic acid levels have been shown to have an approximately 50% lower risk of colon or cervical cancer.

At a 40% DNA demethylation level, for humans and other mammalian species, degenerative death tends to occur. At this level of DNA demethylation, if generalized throughout the tissues, information integrity is so impaired, that survival of the organism is no longer supported. Thus any factor that slows, stops or reverses the loss of methyl groups from DNA will tend to slow, stop and even reverse the aging process at the DNA level.

Although a 50% DNA demethylation level throughout the body would generally not support survival, a loss at this level can occur in selective tissues in certain conditions. In particular, 50% DNA demethylation has been reported selectively in lymphocyte populations in the autoimmune diseases systemic lupus erythematosis and rheumatoid arthritis.

The extreme loss of DNA information integrity in these immunity regulating cells may be at the core of dysfunction that results in the immune system identifying self antigens as foreign antigens and initiating a destructive inflammatory process against the self. Various anti-inflammatory agents work primarily to reduce the end inflammatory effects rather than address the core information and DNA regulatory defects. In contrast, correcting the methylation defects in affected immune cells may help correct autoimmune conditions at the level of information dysregulation.

Homocysteine elevation, associated with both accelerated DNA demethylation and telomere shortening, is a marker for accelerated aging processes at the DNA level. Any program that intends to achieve life extension effects must address DNA methylation, SAMe generation, and homocysteine levels to be complete.

The pathologic effects of homocysteine extend beyond accelerated DNA demethylation. Homocysteine is also a significant factor in increasing the pathogenicity of cholesterol in the etiology of vascular disease. Homocysteine and thiolactone combine with LDL cholesterol to promote LDL oxidation.

Animal studies have shown that administration of high doses of unoxidized cholesterol has little effect on blood vessels, but the addition of even a trace of oxidized cholesterol results in rampaging atherosclerosis. The action of homocysteine to induce LDL cholesterol oxidation can greatly increase the atherogenicity of LDL cholesterol, even at levels considered to be in the normal range.

In addition, elevated homocysteine increases the binding of lipoprotein(a) to fibrin. Elevated homocysteine also tends to increase the propensity of the soluble clotting factors to form blood clots. Both of these factors increase the likelihood that a blood clot will form and obstruct a vessel, especially in a region of vulnerable vascular plaque, that may result in a heart attack, stroke, or peripheral tissue gangrene.

Studies of blood vessel tone show that the higher the homocysteine level above a physiologic normal, the greater the inhibition of nitric oxide production from the vascular endothelium. As nitric oxide dilates blood vessels, inhibiting nitric oxide impairs the ability of the affected vessel to dilate in response to a need for greater blood flow. Antagonism of nitric oxide production may predispose to vascular spasm, increasing the likelihood that a tissue will undergo ischemia, or reduced blood flow and oxygenation below that needed to support viability of the tissue.

Through these multiple mechanisms, elevated homocysteine may accelerate atherosclerosis, impair blood vessel dilation required for adequate blood flow, or increase the likelihood of blood clot formation. For these reasons, homocysteine can be a much greater risk factor for premature heart attack (below age 55) then elevated cholesterol, as well as for stroke and peripheral vascular disease. Elevated homocysteine has been shown to increase the relative risk of a premature heart attack by up to 40 fold, whereas the relative risk for increased cholesterol is only about 4 fold.

From the standpoint of malignancy, homocysteine has been found to accumulate in malignant cells and interfere with DNA and protein chemistry. Administrating methylation enhancing nutrients to smokers with premalignant bronchial cytology showed a significant regression of lesions toward normal, whereas there was no improvement in bronchial cytology of the placebo control group. In addition, administering methylation enhancing factors has also appeared to improve the clinical course of lymphoma.

Reducing homocysteine levels and improving methyl metabolism may have wide ranging benefits, including anti-aging effects, reducing cardiovascular risks, and reducing the risk of and mitigating the course of malignancy. Elevating SAMe has also been associated with relieving depression and osteoarthritis symptoms, improved symptom profiles in fibromyalgia, and enhancement of cardiac and liver health and function.

A randomized placebo controlled double blind prospective clinical study was performed to assess the effects of laser treated betaine plus laser treated cofactors on homocysteine levels and other clinical and metabolic profiles. A comprehensive protocol for human clinical study was submitted to the Western Institutional Review Board (WIRB) in Olympia, Wash., which protocol was approved for following accepted guidelines for human clinical studies.

Study subjects were recruited from the Seattle and Olympia, Wash. areas through notification in a local newspaper. Forty subjects over the age of forty were selected for participation. The minimum age of forty was selected as homocysteine levels tend to rise with age, to choose a study group expected to have at least a moderate level of homocysteine elevation to see the effects of the study formula on reducing homocysteine levels.

The study formula, designed and laser treated for enhancement of methylation metabolism, consisted of the following ingredients in the stated proportions: betaine (trimethylglycine) 2000 mg, choline bitartrate 750 mg, inositol 500 mg, inositol hexanicotinate 375 mg (a nonflushing form of niacin, vitamin B3, that provides 80% niacin by weight, or 300 mg of niacin), magnesium amino acid chelate 18.42% 162.9 mg (providing 30 mg magnesium), cyanocobalamin 1% 100 mg (providing 1 mg of vitamin B12), pyridoxine hydrochloride 25 mg (vitamin B6), zinc chelate 20.17% 24.8 mg (providing 5 mg zinc as amino acid chelate), calcium chloride 37 mg, magnesium stearate 27 mg, pyridoxal-5-phosphate 5 mg (vitamin B6 in phosphorylated form), and folic acid 1.6 mg. These ingredients were measured by weight and mixed to a uniform consistency and distribution in a commercial mixing device. The total weight of the formula, balanced to 2 grams of betaine is 4.008 grams, filling 6 gelatin caps of 00 size with 668 mg each.

The study methylation enhancement formula was treated with sparse constructive node laser illumination at a primary laser wavelength of 670 nm. Two GaAs diode lasers were used with primary powers of 4.6 mW and 3.0 mW phase cancelled to 2.3 mW and 1.5 mW, respectively. These lasers were further electronically modulated at 10 MHz. The study formula was placed in a clear plastic container with 2 kg of formula per container. Each container was treated with dual laser irradiation with the container rotating in a gyroscopic device for 12 minutes per container. The average laser irradiation dose was 0.044 kg/min/mW.

Study subjects were randomized into treatment or placebo groups after entry into the study. Baseline homocysteine levels were stratified by level from high to low, and for each range two thirds of subjects were randomized to receive active treatment with the laser treated methylation formula and one third to receive a placebo.

All subjects received, reviewed and signed informed consent forms for the study protocol before starting the study. All subjects ingested 18 cobalt blue opaque gelatin capsules that obscured for the subjects whether they were receiving placebo or active formula. During the first month of the study, the treatment group received 2 grams of laser homogenized betaine plus a proportionate level of laser treated cofactors daily; the balance of the weight of their capsules was filled with maltodextrin 580, a low glycemic carbohydrate polymer of glucose that would not be expected to have a significant impact on methyl metabolism.

During the second month of the study the treatment group received 4 grams of laser homogenized betaine plus a proportionate level of laser treated cofactors daily; the balance of the weight of their capsules was filled with maltodextrin. During the third month of the study the treatment group received 6 grams of laser homogenized betaine plus a proportionate level of laser treated cofactors daily; this quantity of formula completely filled the capsules and no additional maltodextrin was required. For the entire duration of the study, all of the capsules ingested in the placebo group were filled only with maltodextrin.

All subjects filled out daily questionnaires that indicated the number of capsules they ingested on each day. On the daily questionnaires subjects also recorded food and beverages ingested, amount of exercise, whether they smoked and how much, as well as their general mood, energy, and quality and duration of sleep. In addition, space was provided to record any symptomatic benefits, side effects or general comments.

Intake and study completion surveys were also done just before starting and upon concluding the study. In addition to the general dieting, exercise, well being, and smoking questions asked above, these surveys also inquired about the presence of any known medical problems, medication use, nutritional supplement ingestion, and any alcohol or caffeine intake.

At baseline, and after each week of the study, all subjects completed a clinical assessment questionnaire known as the SCL-90-R. Produced by National Computer Systems, Inc (NCS), SCL-90-R stands for Symptom Checklist 90-Revised. The SCL-90-R is an extensively used highly statistically validated survey of 90 questions used in “clinical trials to help measure the change in symptoms such as depression and anxiety.” It is a brief multidimensional self-report inventory that screens for symptoms of psychopathology and provides global distress indices. NCS provides a scoring template that gives a percentile rank for the study subject for each of the symptom scales tested, for the study subject compared to the general population. Test scales measured included anxiety, depression, paranoid ideation, obsessive-compulsive, somatization (perceptions of bodily dysfunction), and hostility scales, and a global severity index, the latter an index for all symptoms measured together as a composite.

At baseline, and at the end of each month of the study, subjects reported to the clinical laboratory of St. Peters Hospital in Olympia, Wash. for phlebotomy. Blood tests measured serially were complete blood counts with differential white blood cell counts and platelets, chemistry panels including glucose, electrolytes, blood urea nitrogen (BUN), creatinine, liver enzymes, and lipid panels that included triglycerides, total cholesterol, LDL cholesterol, and HDL cholesterol. Homocysteine levels were also drawn. In addition, blood samples were centrifuged and fractionated for red cells, white cells, and plasma components and then frozen for specialized studies to be conducted at an independent research laboratory, to include red blood cell SAMe and DNA methylation levels. Samples were collected and shipped as per the established lab protocol.

For all areas measured, not more than ten subjects dropped out of any measure. The main reasons for dropping a subject from data analysis were failure to do one or more blood tests or complete forms, a medical or metabolic condition interfering with analysis, or other administrative reasons.

As this study achieved baseline and three different dosage measures, the statistical analysis method of autoregression with multiple measures was employed. As this method uses each subject as their own control, a formal control group is not required for statistical analysis. The placebo control group was used in this study primarily to exclude significant random fluctuations in the metabolic measures tested in the absence of the study formula.

The reduction of homocysteine level was statistically significant at every dosage given, with p<0.00001 even at the lowest dose. The average homocysteine level in the treatment group dropped from 9.1 at baseline to 7.1 after the first month of the study formula, using 2 grams of laser homogenized betaine plus laser treated cofactors. Reductions in the treatment group at the second and third dosage levels of 4 grams and 6 grams of homogenized betaine plus proportionately increased levels of laser treated cofactors yielded average homocysteine values of 6.8 and 6.1, respectively. At 6.1, this placed the treatment group as a whole at the lowest cardiovascular risk level for homocysteine, below that of the general population.

FIG. 6B shows the dose response curve graphically with the statistical significance values for each dosing level.

The placebo control group started at homocysteine levels not statistically significantly different from the treatment group. Over the 3 month course of the study there was no significant reduction in homocysteine levels; if anything there was a minor statistically insignificant increase in homocysteine levels. The average homocysteine values for the placebo control group over the three months of the study are shown graphically in FIG. 6C.

As greater degrees of homocysteine elevation are associated with commensurately higher cardiovascular and other risks, the subgroup of the treated subjects who started with the highest homocysteine levels was separately analyzed for dose response effects.

FIG. 6D shows the dose response curve to the laser treated study formula for those subjects whose baseline homocysteine values were at least 10. The average reduction was statistically significant at every dosage level, with a 30% reduction from 13.2 to 9.3 even at the lowest dose of the study formula. Higher doses further reduced the homocysteine levels on average to 8.3 and 7.3, after the second and third months, respectively. The highest proportionate drop was a subject whose baseline homocysteine of 15 dropped to 5 after the second month of the study formula, or a nearly 70% reduction of homocysteine. These results indicate that the laser homogenized methylation formula may be especially helpful for regulating and lowering the highest risk elevations of homocysteine.

The use of the study formula was also associated with a statistically significant reduction of the anxiety scale. FIG. 6E shows the linear dose response curve for greater reduction of anxiety with higher doses of the study formula. In contrast, there was no significant reduction of anxiety scale in the placebo group.

FIG. 6F shows a highly significant reduction of the somatization scale (perceptions of bodily distress, aches and pains) with an especially steep reduction at the lowest dosage level.

FIG. 6G shows statistically significant reduction of depression, increased at higher dosage levels. As the study formula is expected to increase SAMe levels, the results shown in FIGS. 6F and 6G are consistent with the reported effects of the use of SAMe directly—namely reductions of aches and pains, whether due to osteoarthritis or fibromyalgia, as well as relief of depression.

FIG. 6H shows the highly statistically significant reduction of obsessive-compulsive symptoms at every dosage level.

FIG. 6I shows a significant and linear reduction of paranoia symptoms with increasing doses of the study formula.

FIG. 6J shows a statistically significant reduction of hostility with use of the laser homogenized study formula. Recent research has shown a correlation with elevated homocysteine and increased human hostility. This is one of the first interventions to show not only a reduction in homocysteine, but also a corresponding reduction in measured hostility.

FIG. 6K shows the dose response curve for the global severity index, an overall measure of all the symptom and severity scales assessed collectively. This index shows highly statistically significant reductions of the global symptom profile at all dosage levels, increasing at every dose, with an especially marked relative response at the lowest dose.

Improving fundamental methyl group transfer biochemistry, especially at the level of cell membrane fluidity and function, neurotransmitter production and balance (particularly of serotonin), post-transcriptional modification of proteins, DNA synthesis and repair, endothelial vascular protection, and numerous other facilitated pathways may be expected to have widespread benefits on cellular metabolism and function.

The optimum use of the laser homogenized methylation formula can be adjusted based on the response to treatment of homocysteine levels, SAMe levels, DNA methylation assays, inflammatory markers, or changes in clinical condition. In persons who are clinically well, it would be advised to adjust the dosage of the formula to sustain the homocysteine levels associated with the lowest cardiovascular risk, at or below the cutoff value of 6.3.

### EXAMPLE9

### Case Report of Improved Lupus with Laser Treated Methylation Formula as a Model for Relief of Autoimmune Disease Pathology

Autoimmune disease is a condition in which the immune system recognizes self-antigens as foreign and initiates an immune inflammatory attack on self-tissues. Central to the disease process is an information defect in the ability of the immune system to distinguish components of host tissue from foreign or invading antigens.

A phenomenon repeatedly observed in two of the most common autoimmune diseases, lupus and rheumatoid arthritis, is extensive DNA demethylation of T cell lymphocytes. Although lymphocytic DNA demethylation could be a phenomenon secondary to the inflammatory response, it is also possible that the DNA demethylation process has a primary role in disease etiology through impaired regulation of DNA control mechanisms. Recent research showing that clinical improvement in rheumatoid arthritis with methotrexate treatment is associated with increased DNA methylation supports the hypothesis of DNA demethylation as an etiologic factor in disease.

If the tissues of the body suffer a generalized 40% DNA demethylation, degenerative death usually occurs. In rheumatoid arthritis and lupus, up to 50% DNA demethylation of T cell lymphocytes is observed, suggesting an accelerated degenerative process selective to these immune regulatory and effector cells. Aggressive treatment to remethylate DNA may do more than merely suppress the inflammatory response secondary to immune dysregulation, but may help to relieve and correct the underlying information defect at the DNA level.

The exemplary patient, a 59-year-old white female, had suffered with relapsing lupus for several years. At the time of entry into the methylation formula studies of EXAMPLE 6, she had experienced a relapse of severe disease for several months.

Her disease was characterized by exquisitely tender blistering and ulcerating lesions on her hands and feet that made it difficult to walk or open a cabinet door without severe pain. Her skin was pallid, she was extremely fatigued with a chronic low energy state, and had suffered extensive hair loss. Her sedimentation rate, a marker for systemic inflammation, was highly elevated at 99, whereas a normal level would be 0-30. She was treated only with Plaquenil that did little to relieve her symptoms. She declined the use of corticosteroids due to severe treatment side effects during a prior relapse.

The subject was randomized to the placebo control group and for 3 months had no improvement in condition. In the second phase of the study, she was placed on a high dose of the methylation formula of 6 grams of laser treated betaine plus cofactors daily. Within 1-2 weeks of starting the active formula she began to notice clinical improvement.

At the conclusion of the 3 months of the second phase, she reported clearance of 90% of the lesions on her hands and feet with a marked improvement of her malaise and fatigue. At this time her sedimentation rate had dropped to 58. She remained on the same dose of Plaquenil for the course of the study, her only change in treatment being the addition of the active methylation formula in the second 3-month phase of the study.

The subject continued on a lower dose of the laser treated methylation formula for 5 more months, reduced to 1-2 grams of the laser treated betaine plus cofactors. During this period she had complete remission of all clinical symptoms. At the end of fifth month of lower dose treatment, her sedimentation rate had dropped to the very low normal value of 1, the lowest level ever recorded for her.

Other clinical markers indicated significant improvement in her underlying lupus. Pretreatment C-reactive protein was elevated at 3.4 (normal 0-1.5) that decreased to normal at 1.1 at the end of the treatment course.

Pretreatment complement levels of C3 and C4 were reduced to 84 (normal 94-192) and 11.5 (13.0-52.0), respectively, indicating an active inflammatory process consuming complement factors. By the third month of the high dose methylation formula, her levels had returned to normal with a C3 and C4 of 98 and 13.2, respectively, demonstrating a reduction in the autoimmune inflammation. Anti-double stranded DNA antibodies are a specific marker for SLE. Pretreatment titers elevated at 1:40 dilution (normal <1:10 dilution) were reduced essentially to normal at 1:10 dilution by the end of the first month of use of the high dose formula.

She had complete clearance of the lesions on her hands and feet and complete resolution of all other clinical features. Her energy returned to a high level for the first time in several years. Her pallor resolved and her hair regrew luxuriously.

She discontinued the use of the methylation formula at this time and felt clinically well for 7 months. However, a repeat sedimentation rate after 7 months off the formula showed an increase of her level to 103. Within a few weeks of the noted sedimentation rate elevation she began to have aching in her fingers and the early onset of skin lesions in her hands. Within a few weeks of resuming the high dose laser treated methylation formula, her early recurrence symptoms resolved fully. With continued use of the formulation her sedimentation rate again returned to normal.

Her experience is consistent with numerous studies in methylation chemistry that indicate a prompt tendency of the biochemical markers to return to pretreatment levels after stopping delivery of methylation enhancing factors. In general, long-term consistent use is recommended for the best results.

Systemic lupus erythematosus is a prime example of a wide range of autoimmune conditions with immunologic attack on self-antigens. Aggressive remethylation with the laser treated methylation formula is an appropriate treatment to consider for any form of autoimmune disease, especially those known to be characterized by reduced lymphocyte methylation, such as rheumatoid arthritis and lupus. Such treatment has a very high therapeutic index and may help remedy underlying DNA regulatory defects rather than merely suppress symptoms due to the inflammatory process.

### EXAMPLE 10

### Potential Prion Inactivation and Other Protein Reshaping Effects Using Laser Acoustic Resonance

Prions are a unique class of proteinaceous infectious agents particularly noted for causing slowly progressive neurodegenerative disease. Prions are distinct from other classes of transmissible agents in that they do not require DNA or RNA effector mechanisms to cause pathological changes. Prions have been observed to pass through microfilters too small in pore size to admit even the smallest viruses or bacterial agents. They are also resistant to sterilization at temperatures usually effective for clearing microbial pathogens. With a deceptive biologic strategy independent of nucleic acids, no treatment has yet been developed for these devastating disease conditions.

The human syndrome most closely associated with prion transmission is Creutzfeld-Jacob disease. Although rare, Creutzfeld-Jacob disease is the most common spongiform encephalopathy in humans, characterized by typical vacuolar changes in brain tissue and astrocyte proliferation. Disease transmission has been reported through injection of growth hormone prepared from pooled human pituitary extracts, corneal transplantation, and implantation of contaminated stereotactic electrodes to treat epilepsy. Incubation periods have typically ranged from 15-31 months. The average duration of illness is approximately 6 months to demise from progressive dementia, myoclonus, and motor dysfunction.

As defined by Prusiner, prions are “small proteinaceous infectious particles which resist inactivation by procedures that modify nucleic acids”. Perhaps the most extraordinary feature of this class of diseases is that the pathological protein appears to be encoded by the host cell genome. The gene for the human prion protein (PrP) has been mapped to chromosome 20. The normal gene product, PrP^{c}, appears to have the same amino acid sequence as the pathological protein, PrP^{sc}. Differences in the 3-dimensional folding convert the normal variant of the membrane sialoglycoprotein to an abnormal isoform that aggregates into nodes of pathological proteins visible with electron microscopy. Prion aggregates may be responsible for the amyloid plaques and fibrils seen in brain tissue in this group of diseases.

Chaperonins are a class of effector proteins that help to shape peptide sequences into their biologically active 3-dimensional conformation. A dysfunction of chaperonin activity in the prion diseases may be responsible for the abnormal folding and aggregation of the otherwise normal peptide sequences.

Applying the ‘bang and ring’ of sparse constructive nodes, while orienting and shaping molecules with their relatively large EM field waves compared to molecular size may provide chaperonin-like effects. The sparse constructive node and EM field patterns may help guide the 3-dimensional folding of peptide sequences, mimicking the process of shaping in the chaperonin pocket. The ability of sparse constructive node irradiation to modify the shape of individual amino acids may thus be potentially extended to chains of amino acids to favor desirable polypeptide folding patterns.

To determine whether laser acoustic resonance may be able to favor the normal as opposed to the pathological conformation of PrP, we would do acoustic spectrum analysis of both forms. If the acoustic spectra of the preferred to non-preferred form are different, then in principle there would be the possibility of favoring the formation of the desired form by applying sparse constructive node laser irradiation modulated with the spectral frequencies of the preferred form. Even then, it is possible that the total energy required to switch from one form to the other may be well above that achievable by resonance before other damping losses dominate.

A cost effective method for determining acoustic resonance spectra for application to complex molecules would use sonoluminescence with supersaturated carbon dioxide bubble nucleation to create a single point acoustic emitter in a solution. The main example of sonoluminescence is the use of ultrasound to compress small bubbles to infinitesimal size, resulting in a sudden dramatic increase in temperatures sometimes by many thousands of degrees in a tiny space.

In some systems this temperature spike (often with light) can be used to drive chemical reactions directly; however, in this context the bubble nucleation is used to create a single point acoustic emitter that can be used to measure acoustic absorption spectra of molecules in solution.

For example if carbon dioxide is dissolved in water using a ‘soda stream’, and you place in the water a wide band hydrophone made of PVDF (polyvinyledene fluoride), the acoustic spectrum of pure water will be measured. Dissolving a test molecule of choice in the water will change the absorption spectrum. The differential absorption spectrum will show the frequencies of the main modes of oscillation of the molecules tested. In addition, the narrowness of the absorption lines will show the homogeneity of the compound in solution.

In the general case of use, we would choose the largest absorption line and tune the laser modulation to that frequency. Such primary resonant frequencies can be delivered highly efficiently to the molecules. This will provide an additional level of control of molecular shapes over and above the general “bang and ring” effect of using the dirac-like acoustic spikes of photon absorption and re-emission.

In a simple lab experiment of homogenization, a dry powder (or solution) of a compound could be divided into two batches. One batch would be irradiated with a modulation frequency chosen from a previous CO₂ nucleation absorption spectrum analysis. This powder is then added to a CO₂ solution and the control powder is added to a different CO₂ solution. The absorption spectrum of each of the two solutions is then to be measured. To the degree homogenization has occurred, the irradiated sample will show a narrower absorption spectrum than the control sample.

A simple compound like betaine will show a relatively small number of absorption lines, while a compound like fibronectin or glucoamylase will have hundreds. Each line chosen for irradiation is expected to narrow after irradiation.

For the prion example, the normal and pathological prion configurations would be dissolved in separate CO₂ solutions and the absorption spectrum of these solutions would be measured. Absorption peaks seen in the normal versus pathological prions could then be replayed into the pathological prion solution to favor the resonances and configurations of the normal form. The frequencies would be applied as modulations of a beam of sparse constructive nodes of laser acoustic resonance.

Conversely, absorption peaks of pathological prions could be replayed into the solution of the pathological forms to heat the local resonances sufficiently to disrupt the overall structure. Such intense local heating may simply denature the 3-dimensional conformation or, if targeted to susceptible bonds, may cause disruption of covalent bonds. The primary laser wavelength, if intended for resonant denaturation, would be shifted toward the violet-ultraviolet end of the electromagnetic spectrum, whereas the infrared-red end of the spectrum is more suited to the reconfiguration strategy.

The ability to convert pathological prions to a normal configuration or to denature the structure would have a potential role as a rapid low energy sterilization procedure for tissues or instruments for transplantation. Because of the increased depth of penetration of sparse constructive nodes through tissue compared to ordinary conventional laser EM irradiation, this energy could also be applied as a direct in vivo treatment. The clearance of pathological prions from clinical samples, tissues, and instruments for greater lab and clinical safety could also be accomplished.

If further developed, there are also potential veterinary and animal husbandry applications. Prion disease in animals causing spongiform encephalopathy is especially well recognized in sheep and goats as scrapie and in cows as mad cow disease. Applications could include treatment or prevention of this otherwise untreatable disease or to sterilize potentially infected or infectious tissues or contaminated instruments.

From a general applications standpoint, the process of using sonoluminescent CO₂ nucleation absorption spectral analysis can provide resonant modulation frequencies to further enhance the intended homogenization effects. Other spectrographic methods may be used, but the advantage of this suggested preferred mode is its ease and cost effectiveness.

Modulating sparse constructive nodes of laser irradiation with resonant spectral peaks may cause further specific structural changes over and above the general homogenization and flattening effects. This may be especially important for enhancing desirable effects of pharmaceuticals, especially agents targeted to receptor effects. This may further improve receptor shape fit, increase desirable therapeutic action at a given dose, and reduce non-specific dose related and dose-independent adverse effects.

Such further specific targeting of a wide range of nutraceutical and pharmaceutical effects would require further in vitro, animal and clinical testing as appropriate to the desired effect. A prime candidate for such effects would be modifying the action of agents that function in the receptor pathways of the phenolic neurotransmitters dopamine, epinephrine, and norepinephrine. The ability to modify the backbone twist and overall flattening and shape of molecules with phenol (hydroxylated benzene) rings may enhance desired function and reduce the often significant side effects.

Virtually all receptor-ligand and enzyme-substrate mediated systems are highly shape dependent. The ability to modify and homogenize ligand or substrate shape will concentrate the effect of the shape modification either to increase or decrease reactivity of the system as desired. Thus, a wide range of nutrients, pharmaceuticals, and other bioactive agents may be modified to enhance the intended biological or physiological effects.

Specific resonances using higher frequency blue-violet to ultraviolet primary laser systems may be found that denature specific pathological agents. Using modulation of sparse constructive laser irradiation may potentially inactivate a wide range of pathogens.

Specific resonance systems may greatly raise the temperature of selected chemical bonds, making them more reactive. Some covalent bonds may be susceptible to breakage, resulting in a reactive fragment of specific shape and structure. This may be used to create reaction sequences that would otherwise be thermodynamically unfavorable to increase yield of structures difficult to produce or to create novel beneficial compounds.

Potential development of the use of sparse constructive node resonant laser irradiation thus includes the possibility of reshaping prions to render them no longer pathogenic; enhancing or even mimicking intrinsic enzyme, receptor, and signal transduction systems; and modifying components of a wide range of infectious agents or toxins to reduce their pathogenicity or toxicity.

### EXAMPLE 11

### Clinical Effects of Laser Treated L-Arginine to Reduce Blood Pressure and Cholesterol Levels

Subjects in the double-blind study of EXAMPLE 6, upon completion of the first phase of the study, were invited to participate in the Western IRB approved second phase of the study. Subjects that had been in the placebo group were invited to enter a dose-response study of a laser treated L-arginine formula. Subjects that had been in the active treatment group were invited to continue with the high dose methylation formula while adding a dose-response study of a laser treated L-arginine formula.

Each size 00 capsule of the laser treated L-arginine formula was compounded to provide 500 mg of homogenized L-arginine. In addition, each capsule contained the following composition of laser treated ingredients: inositol hexanicotinate 25 mg (80% molar ratio of niacin, or 20 mg of niacin), pyridoxine (vitamin B6) 2.5 mg, magnesium amino acid chelate 18.42% 54.3 mg (providing 10 mg of magnesium), zinc amino acid chelate 20.17% 4.1 mg (providing 0.833 mg of zinc), and selenomethionine 0.5% 2.33 mg (providing 11.67 micrograms of selenium chelated to methionine), and calcium pantothenate (vitamin B5) 11 mg.

Laser homogenization of the arginine plus supportive vitamins and mineral cofactors was performed as follows. Dry powder of this formula weighing 2 kg per clear plastic container was placed on a gyroscopic device rotating the product through three axes. Two diode lasers of 670 nm with primary powers of 4.6 mW and 3.0 mW were conjugated into sparse constructive node laser irradiation at 2.3 mW and 1.5 mW, respectively. The beams were also further amplitude modulated at 10 MHz electronically. Average laser dose was 0.044 kg/min/mW for a treatment duration of 12 minutes per container.

Subjects that had been in the treatment group in the first phase of the study were continued at the dose of 6 grams of laser treated betaine plus laser homogenized cofactors, the highest daily dose of the treated methylation formula from the first phase. For the first month, the subjects took 9 capsules daily of the laser treated arginine formula, providing 4.5 grams of activated arginine plus a proportionate ratio of treated cofactors. For the second month, the daily dose was increased to 18 capsules, or a base of 9 grams of laser treated arginine. During the third month, the daily dose was increased to 27 capsules daily, or 13.5 grams, as tolerated by the GI tract.

The placebo group from the first phase of the study was switched to taking the laser treated arginine formula only. For months 1, 2 and 3 of the second phase, the arginine base doses were 4.5 grams, 9 grams, and 13.5 grams daily, respectively, with the same proportion of laser treated cofactors as the comparison group also taking the treated methylation formula.

Three subjects declined taking the activated arginine complex either because of the known tendency of arginine to predispose to recurrences of Herpes simplex viral outbreaks, or due to research suggesting it be used with caution in persons with autoimmune disease. One of these participants was the subject with active lupus whose course is described in EXAMPLE 7. All subjects in this group were given the highest dose of the laser treated methylation formula studied, 6 grams of treated betaine plus proportionate cofactors.

Subjects continued to complete daily questionnaires as described in EXAMPLE 6, in particular to document daily capsule ingestion of the respective capsules, as well as for reporting subjective benefits or side effects. Weekly SCL-90 questionnaires were also done. At the end of the study subjects completed exit questionnaires and study summaries.

At baseline and monthly, patients had the following blood work drawn and analyzed: triglycerides and total, LDL, and HDL cholesterol levels, and red cells, white cells, and plasma for advanced studies at an independent research lab. In addition, baseline and monthly, the subjects had their blood pressure measured at the time of the blood draws. An approved informed consent form was provided by Western IRB for the second phase of the study. After review and signature of these informed consent forms, the subjects began the second three-month phase of the study. Over the course of the second phase, subjects taking the laser homogenized L-arginine formula showed statistically significant reductions in total cholesterol, LDL cholesterol, and the ratio of total to HDL cholesterol, with significant reduction in these measures requiring 2-3 months of use. In addition systolic and diastolic blood pressure also showed statistically significant reduction, again requiring 2-3 months of use to achieve significant levels. Triglyceride levels also dropped from an average of 140 to 118, but this reduction did not achieve statistical significance.

In contrast to the first phase of the study in which the recommended intake of capsules was consistently ingested, intake was quite variable in the second phase of the study. Instead of a dose response curve, the results are more indicative of cumulative effects over time of bulk dosage intake for the entire group.

Based on summation of capsule intake from the daily reports, the range of ingestion of the laser treated L-arginine formula for the first month was 105-410 capsules. The average intake was 210 capsules, or approximately 7.0 capsules per day. During the second month, the range of ingestion was 126-513 capsules, with an average intake of 386 capsules, or approximately 12.9 capsules per day. For the third month, the range of ingestion was 27-756 capsules, with an average intake of 436 capsules, or approximately 14.5 capsules per day. The average daily intake of the laser homogenized L-arginine for the first, second, and third months was thus 3.5 grams, 6.5 grams, and 7.3 grams, respectively.

A one-way repeated measures analysis of variance was used to analyze the effect of the laser homogenized L-arginine formula on total cholesterol levels over time, with the analysis limited to the 29 subjects whose baseline total cholesterol exceeded 180. The multivariate tests indicate a significant cholesterol reduction effect, Wilks' Lambda=0.77, F (2,26)=3.813, p=0.035. This effect required 2 months to be evident.

From baseline to the end of the second month, 18 of 29, or 62% of treated subjects showed a reduction in total cholesterol. Of those who showed a reduced total cholesterol, 61% had a reduction of 10% or more, and 18% showed a reduction of 20% or more. The greatest individual total cholesterol reduction of 32% was from a baseline of 213 to a treated level of 146.

A one-way repeated analysis of variance was used to test the effect of the laser homogenized L-arginine formula on LDL cholesterol over time. The multivariate tests indicate a significant LDL cholesterol reduction effect, Wilk's Lambda=0.655, F(3,20), p=0.034. Further paired t-test analysis revealed that the most significant drop in LDL cholesterol occurred after the third month of treatment. The average baseline level was elevated at 140 and dropped after the third month of treatment to 128, a clinically important level of reduction.

Of 26 subjects with a baseline cholesterol over 180 , 61% showed a reduction in LDL cholesterol after 3 months of treatment with laser homogenized L-arginine. Of those that showed a reduction, 75% had a lowering of 10% or more, and 25% had a lowering of 20% or more. The greatest single reduction was 66%, from a highly elevated level of 223 to a normal level of 75.

To assess the relative effect on HDL cholesterol, a one way repeated measures analysis of variance was computed for the dependent variable of the total to HDL cholesterol ratio. The multivariate analysis of variance indicates a significant effect in reducing this ratio over time, Wilk's Lambda=0.691, F(3,21), p=0.048. The average baseline ratio of 4.1 decreased after three months of treatment to 3.8, a 7% reduction, with 63% of subjects showing a reduction in this ratio.

A one-way repeated measures analysis of variance was computed for the dependent variable systolic blood pressure over time with the independent variable being the treatment formula. A statistically significant reduction in systolic blood pressure was found over time, Wilks' Lambda=0.715, F(3,26)=3.447, p=0.03; a significant linear effect was also found, F(1,28)=6.522, p=0.016.

The average systolic blood pressure of 131 for the entire group dropped to 126 after three months of treatment with the laser homogenized L-arginine formula, the paired t-test comparing baseline to three months of treatment statistically significant at p=0.004. In the subgroup of subjects with systolic hypertensive blood pressure of 140 mmHg or higher, 9 of 10, or 90% demonstrated a reduction in systolic blood pressure after three months of study formula treatment, which reduction was statistically significant at p=0.033; pretreatment values in this group ranged from 140-208 mmHg which dropped post treatment to 123-160 mmHg. The range of reduction of systolic blood pressure in those subjects showing a drop was 2-48 mmHg, with an average reduction of 19.4 mmHg.

A one-way repeated measures of variance was computed on the dependent variable of diastolic blood pressure with the independent variable the ingestion of the laser homogenized L-arginine formula taken over a period of three months. A univariate analysis of variance indicated a significant reduction in diastolic blood pressure, F(3,26)=4.014, p=0.01. A significant linear effect in the reduction of diastolic blood pressure was observed, F(1,28)=7.236, p=0.012. Using paired t-tests, statistically significant reductions in diastolic blood pressure were seen from baseline to two months of treatment, p=0.043, and from baseline to three months of treatment, p=0.019.

Average diastolic blood pressure at baseline of 82 dropped progressively at one, two, and three months to 81, 78, and 76, respectively. Although only 5 subjects had diastolic hypertension of 90 mmHg or greater, 80% showed a reduction in diastolic blood pressure, the pretreatment range of 90-128 mmHg dropping after three months of treatment to 60-99 mmHg. In those subjects with diastolic hypertension showing a drop in diastolic blood pressure, the range of reduction was 9-40 mmHg with an average reduction of 25.8 mmHg diastolic.

No subject with low normal systolic or diastolic blood pressure had a reduction in blood pressure to hypotensive levels. A common and potentially serious side effect of other antihypertensive remedies, the laser homogenized formulation of L-arginine was found to be entirely free of this problem.

At higher doses of the laser treated L-arginine, some subjects developed gastrointestinal side effects, in particular diarrhea. The literature indicates that up to 15 grams of untreated L-arginine may typically be ingested daily before the development of loose stools. Although some subjects were able to tolerate 13.5 grams of laser treated L-arginine if taken in divided doses, occasional subjects developed loose stools on as little as 4.5 grams, suggesting generally increased potency of the L-arginine; in all cases, reducing the intake below the individual threshold level for GI symptoms resulted in resolution of symptoms.

In addition, male subjects reported improved rather than impaired erectile function when taking the laser treated L-arginine formula. In contrast to many hypertensive agents that may cause impotence, L-arginine supplementation can often increase sexual function, even reversing impotence in a significant fraction of men studied. This is through the effect of L-arginine derived nitric oxide production stimulating increased cyclic-GMP in the genital tissues, the specific signal resulting in the vasodilation that produces the erectile response.

The use of laser homogenized L-arginine may thus effectively reduce total and LDL cholesterol, while improving the total to HDL cholesterol ratio. It may also safely and effectively reduce systolic and diastolic blood pressure. Side effects are relatively few and minor, and usually readily reversed with dose reduction.

Thus, the present invention is able to improve the bioavailability of nutrients, pharmaceutical agents and other bioactive compounds in a mammalian body by treating the compounds with a laser to modify the compound's average structure. The improved bioavailability may be achieved by increasing the absorption of the compound, by decreasing inflammation or other negative reactions to the compound, or by increasing the availability of functional groups to be used in biological processes within the body. Furthermore, because the treatment can be done to the compound in either dry or solution forms, it will be relatively easy for those of skill in the art to modify a wide range of nutrients, pharmaceuticals and other compounds to enhance bioavailability in humans and other mammals.

Thus there is disclosed an improved method for administering dietary amino acids, pharmaceutical agents, and other bioactive substances. While the present disclosure discloses a variety of substances, those skilled in the art will appreciate numerous other substances can be modified within the teachings of the present invention without departing from the scope and spirit thereof. The appended claims are intended to cover such modifications.

What is claimed is:

1. A method for treating anxiety comprising:
preparing modified trimethylglycine and cofactors by subjecting said trimethylglycine and cofactors to laser radiation; and ingesting an effective amount of said modified trimethylglycine and cofactors.

2. The method for treating anxiety according to claim 1, wherein said method comprises consuming at least 2 grams of modified trimethylglycine and cofactors daily.

3. The method for treating anxiety according to claim 1, wherein said method comprises consuming at least 4 grams of modified trimethylglycine and cofactors daily.

4. The method for treating anxiety according to claim 1, wherein said method comprises consuming at least 6 grams of modified trimethylglycine and cofactors daily.

5. The method for treating anxiety according to claim 1, wherein the method comprises forming said modified trimethylglycine and cofactors by exposure to laser radiation with an amplitude modulation at a resonance frequency of one or more acoustic vibration frequencies of said trimethylglycine and cofactors and said laser radiation is structured in polarization and wave patterns.

6. A method for treating depression comprising:
preparing modified trimethylglycine and cofactors by subjecting said trimethylglycine and cofactors to laser radiation; and
ingesting an effective amount of said modified trimethylglycine and cofactors.

7. The method for treating depression according to claim 6, wherein said method comprises consuming at least 2 grams of modified trimethylglycine and cofactors daily.

8. The method treating depression according to claim 6, wherein said method comprises consuming at least 4 grams of modified trimethylglycine and cofactors daily.

9. The method for treating depression according to claim 6, wherein said method comprises consuming at least 6 grams of modified trimethylglycine and cofactors daily.

10. The method for treating depression according to claim 6, wherein the method comprises forming said modified trimethylglycine and cofactors by exposure to laser radiation with an amplitude modulation at a resonance frequency of one or more acoustic vibration frequencies of said trimethylglycine and cofactors and said laser radiation is structured in polarization and wave patterns.

11. A method for treating obsessive-compulsive symptoms comprising: preparing modified trimethylglycine and cofactors by subjecting said trimethylglycine and cofactors to laser radiation; and
ingesting an effective amount of said modified trimethylglycine and cofactors.

12. The method for treating obsessive-compulsive symptoms according to claim 11, wherein said method comprises consuming at least 2 grams of modified trimethylglycine and cofactors daily.

13. The method for treating obsessive-compulsive symptoms according to claim 11, wherein said method comprises consuming at least 4 grams of modified trimethylglycine and cofactors daily.

14. The method for treating obsessive-compulsive symptoms according to claim 11, wherein said method comprises consuming at least 6 grams of modified trimethylglycine and cofactors daily.

15. The method for treating obsessive-compulsive symptoms according to claim 11 wherein the method comprises forming said modified trimethylglycine and cofactors by exposure to laser radiation with an amplitude modulation at resonance frequency of one or more acoustic vibration frequencies of said trimethylglycine and cofactors and said laser radiation is structured in polarization and wave patterns.

16. A method for treating paranoia comprising:
preparing modified trimethylglycine and cofactors by subjecting said trimethylglycine and cofactors to laser radiation; and ingesting an effective amount of said modified trimethylglycine.

17. The method for treating paranoia according to claim 16, wherein said method comprises consuming at least 2 grams of modified trimethylglycine and cofactors daily.

18. The method for treating paranoia according to claim 16, wherein said method comprises consuming at least 4 grams of modified trimethylglycine and cofactors daily.

19. The method for treating paranoia according to claim 17,
wherein said method comprises consuming at least 6 grams of modified trimethylglycine and cofactors daily.

20. The method for treating paranoia according to claim 16, wherein the method comprises forming said modified trimethylglycine and cofactors by exposure to laser radiation with an amplitude modulation at a resonance frequency of one or more acoustic vibration frequencies of said trimethylglycine and cofactors and said laser radiation is structured in polarization and wave patterns.

21. A method for treating hostility comprising:
preparing modified trimethylglycine and cofactors by subjecting said trimethylglycine and cofactors to laser radiation; and ingesting an effective amount of said modified trimethylglycine and cofactors.

22. The method for treating hostility according to claim 21, wherein said method comprises consuming at least 2 grams of modified trimethylglycine and cofactors daily.

23. The method for treating hostility according to claim 21, wherein said method comprises consuming at least 4 grams of modified trimethylglycine and cofactors daily.

24. The method for treating hostility according to claim 21, wherein said method comprises consuming at least 6 grams of modified trimethylglycine and cofactors daily.

25. The method for treating hostility according to claim 21, wherein the method comprises forming said modified trimethylglycine and cofactors by exposure to laser radiation with an amplitude modulation at a resonance frequency of one or more acoustic vibration frequencies of said trimethylglycine and cofactors and said laser radiation is structured in polarization and wave patterns.

26. A method for treating perceptions of bodily distress, aches, and pains comprising: preparing modified trimethylglycine and cofactors by subjecting said trimethylglycine and cofactors to laser radiation; and ingesting an effective amount of said modified trimethylglycine and cofactors.

27. The method for treating perceptions of bodily distress, aches, and pains according to claim 26, wherein said method comprises consuming at least 2 grams of modified trimethylglycine and cofactors daily.

28. The method for treating perceptions of bodily distress, aches, and pains according to claim 26, wherein said method comprises consuming at least 4 grams of modified trimethylglycine and cofactors daily.

29. The method for treating perceptions of bodily distress, aches, and pains according to claim 26, wherein said method comprises consuming at least 6 grams of modified trimethylglycine and cofactors daily.

30. The method for treating perceptions of bodily distress, aches, and pains according to claim 26, wherein the method comprises forming said modified trimethylglycine and cofactors by exposure to laser radiation with an amplitude modulation at resonance frequency of one or more acoustic vibration frequencies of said trimethylglycine and cofactors and said laser radiation is structured in polarization and wave patterns.

31. A method for treating autoimmune disorders comprising: preparing modified betaine and cofactors by subjecting said betaine and cofactors to laser radiation; and ingesting an effective amount of said modified betaine and cofactors.

32. The method for treating autoimmune disorders according to claim 31, wherein said method comprises consuming at least 6 grams of laser treated betaine plus cofactors daily for an induction period of 2-3 months, followed by a maintenance dose of 1-2 grams of said laser treated betaine plus cofactors daily to be maintained or adjusted based on clinical or biochemical response.

33. The method for treating autoimmune disorders according to claim 31, wherein the method comprises forming said modified betaine plus cofactors by exposure to laser radiation with an amplitude modulation at a resonance frequency of one or more acoustic vibration frequencies of said betaine plus cofactors and said laser radiation is structured in polarization and wave pattern.

34. A method for modifying amino acids to reduce the immune reaction to said amino acids, as would be beneficial to provide systemic and tissue amino acids in inflammatory, autoimmune, and allergic conditions, comprising: preparing modified amino acids by subjecting said amino acids to laser radiation; and ingesting an effective amount of said modified amino acids.

35. The method for modifying amino acids to reduce the immune reaction to said amino acids according to claim 34, wherein the method comprises forming said modified amino acids by exposure to laser radiation with an amplified modulation at a resonance frequency of one or more acoustic vibration frequencies of said amino acids and said laser radiation is structured in polarization and wave pattern.

36. A method for modifying amino acids to reduce inflammation, through reducing inflammatory cytokine production in response to said amino acids comprising: preparing modified amino acids by subjecting said amino acids to laser radiation; and ingesting an effective amount of said modified amino acids.

37. The method for modifying amino acids to reduce inflammation, through reducing inflammatory cytokine production, according to claim 36, wherein the method comprises forming said modified amino acids by exposure to laser radiation with an amplitude modification at a resonance frequency of one or more acoustic vibration frequencies of said amino acids and said laser radiation is structured in polarization and wave pattern.

38. A method for improving the quality of crystal formation through increased homogeneity of unit cell elements or reduced defects in the crystal lattice, or both, comprising: selecting the molecular species to be crystallized; and subjecting said molecular species to laser radiation during the process of crystallization.

39. The method for improving the quality of crystal formation through increased homogeneity of unit cell elements or reduced crystal defects or both according to claim 3 8, wherein the method comprises subjecting the selected molecular species, during the crystallization process, to laser radiation with an amplitude modulation at a resonance frequency of one or more acoustic vibration frequencies of said molecular species, and said laser radiation is structured in polarization and wave patterns.

40. A method for improving the quality of crystals that have already solidified through homogenizing unit cell elements and/or liberating trapped water in the crystal lattice comprising: selecting the crystal form to be homogenized and/or dried; and subjecting said crystal form to laser radiation.

41. The method for improving the quality of crystals that have already solidified through homogenizing unit cell elements and/or drying according to claim 40, wherein the method comprises subjecting said selected crystal form to laser radiation with an amplitude modulation at a resonance frequency of one or more acoustic vibration frequencies of the molecular species of said selected crystal form, and said laser radiation is structured in polarization and wave patterns.

42. A method for generating highly crystalline and homogeneous simvastatin comprising: dissolving simvastatin in a solvent and subjecting said simvastatin to laser radiation during the crystallization process.

43. The method for generating highly crystalline and homogeneous simvastatin according to claim 42, wherein the method comprises dissolving said simvastatin in a solvent and subjecting said simvastatin to laser radiation with an amplitude modulation at a resonance frequency of one or more acoustic vibration frequencies of said simvastatin, and said laser radiation is structured in polarization and wave patterns.

44. A method for generating amorphous simvastatin comprising: dissolving simvastatin in a solvent and subjecting said simvastatin to laser radiation during the crystallization process.

45. The method for generating amorphous simvastatin according to claim 44, wherein the method comprises dissolving said simvastatin in ethanol or another solvent and subjecting said simvastatin to laser radiation with an amplitude modulation at a resonance frequency of one or more acoustic vibration frequencies of said simvastatin, and said laser radiation is structured in polarization and wave patterns.

46. A method for modifying the activity of an enzyme, substrate, or ligand, the method comprising: selecting an enzyme, substrate, or ligand to be modified; and subjecting said enzyme, substrate, or ligand to laser irradiation to modify the structure thereof.

47. The method for modifying the activity of an enzyme, substrate, or ligand according to claim 46, wherein the method comprises selecting an enzyme, substrate, or ligand to be modified; and subjecting said enzyme, substrate or ligand to laser radiation with an amplitude modulation at a resonance frequency of one or more acoustic vibration frequencies of said enzyme, substrate or ligand to modify the structure thereof, and said laser radiation is structured in polarization and wave patterns.

48. A method of increasing the depth of penetration of laser electromagnetic signals and energy through tissue to enhance the depth and range of therapeutic efficiency of photodynamic therapy, this method comprising: identifying a condition in tissue that may be responsive to photodynamic therapy; and determining a suitable photodynamic compound, photoactivating laser wavelength, and laser radiation dose to use for treatment of said condition; and administering said photodynamic compound and allowing sufficient time for accumulation of said compound in said tissue to be treated; and applying a sufficient dose of sparse constructive nodes of laser radiation to the tissue to be treated via external beam, endoscopically, endarterially or other route as appropriate, with said laser radiation having an amplitude modulation at a resonant frequency of one or more acoustic vibration frequencies of said photodynamic compound, and said laser radiation is structured in polarization and wave pattern.

49. A method of homogenizing, flattening, and reducing the distortion of backbone twist of aromatic amino acids and L-dopa, and any other dopaminergic, catecholaminergic, or serotonergic precursor, compound, or pharmaceutical agent to enhance the bioavailability of the modified molecular structure, the method comprising: selecting the dopaminergic, catecholaminergic, or serotonergic precursor, compound, or pharmaceutical agent to be modified; and treating said dopaminergic, catecholaminergic, or serotonergic precursor, compound, or pharmaceutical agent with laser radiation.

50. The method of homogenizing, flattening, and reducing the distortion of backbone twist distortion of aromatic amino acids and any other dopaminergic, catecholaminergic, or serotonergic precursor, compound, or pharmaceutical agent to enhance the bioavailability of the modified molecular structure according to claim 49, wherein said method comprises selecting a dopaminergic, catecholaminergic, or serotonergic precursor, compound or pharmaceutical agent to be modified; and treating said dopaminergic, catecholaminergic, or serotonergic precursor, compound or pharmaceutical agent with laser radiation, with an amplitude modulation at a resonance frequency of one or more acoustic vibration frequencies of said precursor, compound or pharmaceutical agent, and said laser radiation is structured in polarization and wave pattern.

51. A method of homogenizing, flattening, and reducing the distortion of backbone twist of a nutrient, pharmaceutical agent, or other bioactive substance to enhance the bioavailability of the modified substance, the method comprising: selecting a nutrient, pharmaceutical agent, or other bioactive substance to modify; and treating said nutrient, pharmaceutical agent, or other bioactive substance with laser radiation.

52. The method of homogenizing, flattening, and reducing the distortion of backbone twist of a nutrient, pharmaceutical agent, or other bioactive substance to enhance the bioavailability of the modified substance according to claim 51, wherein said method comprises selecting a nutrient, pharmaceutical agent, or other bioactive substance to modify; and treating said nutrient, pharmaceutical agent, or other bioactive substance with laser radiation with an amplitude modulation at a resonant frequency at one or more acoustic vibration frequencies of said nutrient, pharmaceutical agent, or other bioactive substance, and said laser radiation is structured in polarization and wave patterns.

53. A method for increasing the bioavailability of nucleic acid bases, nucleosides or deoxynucleosides, or nucleotide or deoxynucleotide monophosphates, diphosphates, or triphosphates, the method comprising: selecting a nucleic acid base, nucleoside or deoxynucleoside, or nucleotide or deoxynucleotide monophosphate, diphosphate, or triphosphate; and subjecting said selected substance to laser radiation to modify the structure thereof.

54. The method for increasing the bioavailability of nucleic acid bases, nucleosides or deoxynucleosides, or nucleotide or deoxynucleotide monophosphates, diphosphates, or triphosphates according to claim 53, wherein the method comprises selecting a nucleic acid base, nucleoside or deoxynucleoside, or nucleotide or deoxynucleotide monophosphate, diphosphate, or triphosphate to modify; and subjecting said nucleic acid base, nucleoside or deoxynucleoside, or nucleotide or deoxynucleotide monophosphate, diphosphate, or triphosphate to laser radiation with an amplitude modulation at a resonance frequency of one or more acoustic vibration frequencies of said nucleic acid base, nucleoside or deoxynucleoside, or nucleotide or deoxynucleotide monophosphate, diphosphate, or triphosphate, and said laser radiation is structured in polarization and wave patterns.

55. A method of increasing the bioactivity of high energy phosphates of nucleotides or deoxynucleotides, the method comprising: selecting a nucleotide or deoxynucleotide to modify; and subjecting said nucleotide or deoxynucleotide to laser radiation.

56. The method of increasing the bioactivity of high energy phosphates of nucleotides or deoxynucleotides according to claim 55, wherein the method comprises selecting a nucleotide or deoxynucleotide to modify; and subjecting said nucleotide or deoxynucleotide to laser radiation with an amplitude modulation at a resonance frequency of one or more acoustic vibration frequencies of high energy phosphates of said nucleotide or deoxynucleotide, and said laser radiation is structured in polarization and wave pattern.

57. A method of increasing the bioavailability of a nucleic acid base, nucleoside or deoxynucleoside, or nucleotide or deoxynucleotide monophosphate, diphosphate, or triphosphate whether or not it has been modified with laser treatment according to claim 128, the method comprising: making a solution of said nucleic acid base, nucleoside, or nucleotide monophosphate, diphosphate, or triphosphate with a concentration at least 10 times that of blood plasma; and applying said solution for at least 30 seconds to oral or other nonintestinal mucosa for direct transmucosal absorption to overcome the extensive degradation of nucleic acid elements as occurs in intestinal mucosa.

58. A method for amplifying or modifying the production or purification of a selected stereoisomer or epimer of a bioactive substance, the method comprising: selecting the stereoisomer to amplify or modify; and subjecting said stereoisomer or epimer to rotationally polarized laser light, with an amplitude modulation at a resonance frequency at one or more acoustic vibration frequencies of said stereoisomer or epimer, and said laser radiation is structured in polarization and wave pattern.

59. A method of reshaping prions or other pathogenic proteins to reduce their pathogenicity, said method comprising: selecting a prion or other pathogenic protein to reshape; and subjecting said prion or other pathogenic protein to laser radiation.

60. The method of reshaping prions or other pathogenic proteins to reduce their pathogenicity according to claim 59, wherein said method comprises selecting a prion or other pathogenic protein to reshape; and subjecting said prion or other pathogenic protein to laser radiation with an amplitude modulation at a resonance frequency of one or more acoustic vibration frequencies of said prion or other pathogenic protein, and said laser radiation is structured in polarization and wave pattern.

61. The method of reshaping prions or other pathogenic proteins to reduce their pathogenicity according to claim 60, wherein said method comprises selecting a prion or other pathogenic protein to reshape; and determining the peak absorption frequencies of said prions or other pathogenic proteins and their nonpathogenic counterparts using sonoluminescence with CO₂ nucleation absorption spectrum analysis or other spectroscopic method or mathematical modeling; and subjecting said prions or other pathogenic proteins to laser radiation with an amplitude modulation of one or more peak absorption frequencies of normal protein, the pathogenic protein, or the differential absorption pattern between the normal and pathogenic counterpart protein to reshape said prions or other pathogenic proteins to reduce their pathogenicity, and said laser radiation is structured in polarization and wave patterns.

62. A method of reshaping pathogenic substances or components of infectious pathogens to reduce their pathogenicity, said method comprising: selecting a pathogenic substance or one or more components of an infectious pathogen to reshape; and subjecting said pathogenic substance or one or more components of said infectious pathogen to laser radiation.

63. The method of reshaping pathogenic substances or components of infectious pathogens to reduce their pathogenicity according to claim 62, wherein said method comprises selecting a pathogenic substance or one or more components of an infectious pathogen to reshape; and subjecting said pathogenic substance or one or more components of said infectious pathogen to laser radiation, with an amplitude modulation at a resonance frequency of one or more acoustic vibration frequencies of said pathogenic substance or of one or more components of said infectious pathogen, and said laser radiation is structured in polarization and wave pattern.

64. The method of reshaping pathogenic substances or components of infectious pathogens to reduce their pathogenicity according to claim 63, wherein said method comprises selecting a pathogenic substance or one or more components of an infectious pathogen to reshape; and determining the peak absorption frequencies of said pathogenic substance or one or more components of said infectious pathogen using sonoluminescence with CO₂ nucleation absorption spectrum analysis or other spectroscopic method or mathematical modeling; and subjecting said pathogenic substance or one or more components of said infectious pathogen to laser radiation, with an amplitude modulation of one or more peak absorption frequencies of said pathogenic substance or of one or more components of said infectious pathogen, and said laser radiation is structured in polarization and wave pattern.

65. A method of selectively activating specific regions of selected molecules to increase the production of desired products in a chemical reaction, to generate novel reaction sequences for products, or to generate the production of novel products with specific molecular shapes, properties, and activities, said method comprising: selecting one or more molecular species to modify; and subjecting said molecular species to laser radiation.

66. The method of selectively activating specific regions of selected molecules to increase the production of desired products in a chemical reaction, to generate novel reaction sequences for products, or to generate the production of novel products with specific molecular shapes, properties, and activities according to claim 65, wherein said method comprises selecting one or more molecular species to modify; and subjecting said molecular species to laser radiation with an amplitude modulation at a resonance frequency of one or more acoustic vibration frequencies of said molecular species, and said laser radiation is structured in polarization and wave pattern.

67. The method of selectively activating specific regions of selected molecules to increase the production of desired products in a chemical reaction, to generate novel reaction sequences for products, or to generate the production of novel products with specific molecular shapes, properties, and activities according to claim 66, wherein said method comprises selecting one or more molecular species to modify; and determining the peak absorption frequencies of said specific regions of selected molecular species to be modified using sonoluminescence with CO₂ nucleation absorption spectrum analysis, other spectrographic method, or through mathematical molecular modeling; and subjecting said molecular species to laser radiation with an amplitude modulation of one or more peak absorption frequencies of said molecular species, and said laser radiation is structured on polarization and wave pattern.

68. A method of selectively activating molecular species or specific regions of molecular species to generate a signal for qualitative or quantitative detection or analysis, said method comprising: selecting a specific molecular species or region of a molecular species to activate through resonance; and subjecting said molecular species to laser radiation with an amplitude modulation at a resonance frequency of one or more acoustic vibration frequencies of said molecular species, and said laser radiation is structured in polarization and wave pattern.

69. The method of selectively activating molecular species or specific regions of molecular species to generate a signal for qualitative or quantitative detection or analysis according to claim 68, wherein said method comprises selecting a specific molecular species or region of a molecular species to activate through resonance; and determining the peak absorption frequencies of said specific molecular species or region of a molecular species using sonoluminescence with CO₂ nucleation absorption spectrum analysis, other spectrographic method, or through mathematical molecular modeling; and subjecting said molecular species to laser radiation with an amplitude modulation of one or more peak absorption frequencies of said molecular species, and said laser radiation is structured in polarization and wave pattern.
